(19) United States
(12) Patent Application Publication
(10) Pub. No.: US 2026/0273191 A1
(43) Pub. Date: Sep. 17, 2026

(54) Wearable Drug Delivery Device For Hazardous Material And Chemical Warfare Agent Detection And Antitode Delivery

(71) Applicant: Ezer Enterprises LLC, Dedham, MA (US)

(72) Inventors: Irving D. Kaplan, Dedham, MA (US); Edward J. Holupka, Medway, MA (US); Howard Zaretsky, Rochester, NY (US)

(73) Assignee: Ezer Enterprises LLC, Dedham, MA (US)

(21) Appl. No.: 19/679,048

(22) Filed: May 15, 2026

Related U.S. Application Data
(63) Continuation-in-part of application No. 18/820,802, filed on Aug. 30, 2024.

(51) Int. Cl. A61M 5/20; A61B 5/00; A61B 5/1455; A61B 5/16; A61M 5/24

(52) U.S. Cl. CPC A61M 5/20; A61B 5/14551; A61B 5/165; A61B 5/681; A61B 5/742; A61B 5/746; A61B 5/7475; A61M 5/24; A61B 2503/20; A61M 2005/2006; A61M 2005/2073; A61M 2205/18; A61M 2205/33; A61M 2230/205

(57) ABSTRACT

A novel and useful wearable drug delivery device and related methods to provide protective medical technologies for use in hazardous environments and to administer therapeutic agents in response to exposure to toxic substances, including chemical warfare agents. The system includes a wearable device that delivers medications to the warfighter on the battlefield. The device is part of a networked, wearable countermeasure platform that integrates directly with the Nett Warrior network to deliver medical countermeasures (MCMs) such as nalmefene and atropine/ipralidoxime chloride in a single ruggedized device. The system delivers the appropriate antidote for nerve agent or opioid aerosol exposure. The system interacts with the warrior so that drug is only delivered when required without the need for self or rescuer administration. It communicates with the Nett Warrior system to provide network-aware capability. The system delivers multiple drugs on the battlefield and is not limited to only one or two.

### REFERENCE TO PRIORITY APPLICATION

This application is a continuation in part of U.S. application Ser. No. 18/820,802, filed Aug. 30, 2024, entitled “Self-Monitoring Based Wearable Apparatus And Related Method For Opioid Overdose Detection And Antidote Delivery,” incorporated herein by reference in its entirety.

### FIELD OF THE DISCLOSURE

The present disclosure relates generally to protective medical technologies for use in hazardous environments, and more particularly to wearable drug delivery systems configured to administer therapeutic drugs/agents or any other type of bioactive substances in response to exposure to hazardous substances including chemical warfare agents.

### BACKGROUND OF THE INVENTION

Military personnel operating in contested and potentially contaminated environments may be exposed to a variety of chemical warfare agents, including, but not limited to, nerve agents, vesicants (blister agents), pulmonary (choking) agents, and blood agents. Such exposure may occur through direct dissemination via munitions, improvised dispersal mechanisms, or secondary contamination of terrain, equipment, or personnel. In many cases, these agents are designed to act rapidly and with high lethality, producing severe physiological effects within seconds to minutes of exposure. For example, certain nerve agents inhibit acetylcholinesterase activity, resulting in the accumulation of acetylcholine at synapses and neuromuscular junctions, leading to convulsions, paralysis, respiratory failure, and, if untreated, death.

Effective medical countermeasures exist for certain classes of chemical agents, including antidotes such as anticholinergics, oxime reactivators, anticonvulsants, and other supportive therapeutics. The efficacy of such treatments, however, is highly time-sensitive, with outcomes strongly correlated to the speed of administration following exposure. Treatment protocols emphasize that antidotes should be administered immediately upon recognition of symptoms, as delays of even a few minutes can significantly reduce survival rates and increase the likelihood of long-term neurological or systemic damage.

Current military doctrine reflects this urgency and relies heavily on decentralized, immediate intervention at the individual warfighter level. Specifically, field manuals and training protocols instruct that, upon recognition of symptoms consistent with chemical agent exposure, a warfighter is expected to perform self-aid by administering one or more antidote auto-injectors carried on their person. In circumstances where the affected individual is unable to self-administer due to incapacitation or severe symptoms, nearby personnel are trained to provide buddy-aid, wherein another soldier administers the antidote using the casualty's own auto-injector kit. Such doctrine further contemplates that additional doses may be administered either by the individual or by a buddy depending on symptom progression, and recognizes that severely affected individuals may be incapable of self-treatment and thus rely entirely on others for timely intervention.

While these self-aid and buddy-aid paradigms provide a critical first line of defense, they present several inherent limitations. Successful implementation depends on rapid recognition of exposure symptoms, availability and accessibility of the auto-injector, and the physical and cognitive ability of the individual or nearby personnel to execute the injection procedure under stressful and potentially hostile conditions. In practice, exposure to certain agents may quickly impair motor control, vision, or consciousness, preventing self-administration. Similarly, in dispersed or high-intensity combat scenarios, a nearby soldier may not be immediately available to provide buddy-aid, or may themselves be affected by the agent. Even when assistance is available, delays in assessing the situation, locating the injector, and performing the injection can materially degrade treatment effectiveness.

Additionally, conventional auto-injector systems are generally passive devices that require manual activation and provide limited flexibility in dosing or timing. They are typically configured for single-use or stepwise administration and do not incorporate sensing, feedback, or decision-support capabilities. Existing systems also lack integration with environmental detection systems or physiological monitoring platforms that could facilitate earlier identification of exposure or more precise tailoring of therapeutic response. They also do not incorporate any connectivity with medical staff, other warfighters, command centers, communication networks, etc. As a result, current approaches create a temporal and functional gap between exposure, detection, decision-making, and treatment.

Furthermore, improper use of antidote auto-injectors, including delayed administration, incorrect dosing, or unnecessary administration in the absence of exposure, may result in suboptimal outcomes or adverse effects. Accordingly, while existing doctrine emphasizes immediate self-aid or buddy-aid as a life-saving measure, it also highlights the operational challenges associated with reliance on manual intervention under conditions of chemical exposure.

In view of the foregoing, there exists a need for improved systems and methods that can provide rapid, reliable, and context-aware administration of therapeutic agents in response to actual or suspected exposure to chemical warfare agents. Such systems may advantageously be embodied in wearable form factors suitable for integration with a soldier's equipment, and preferably incorporate environmental sensing, physiological monitoring, communications, automated or semi-automated drug delivery, and decision logic to reduce reliance on manual activation. By decreasing response latency and mitigating the limitations of self-aid and buddy-aid paradigms, such systems have the potential to significantly improve survivability and operational effectiveness in hazardous environments.

### SUMMARY OF THE INVENTION

The present invention is a novel and useful wearable drug delivery device and related methods to provide protective medical technologies for use in hazardous environments and to administer therapeutic agents/drugs and bioactive substances in response to exposure to toxic or hazardous substances, including chemical warfare agents. The system(s), device(s), method(s), arrangements(s), interface(s), computer program(s), processes, etc., (hereinafter each of which will be referred to as system, unless the context indicates otherwise), described herein address the disadvantages and problems in prior art systems.

The system includes a wearable device that delivers medications to the warfighter on the battlefield. The device is part of a networked, wearable countermeasure platform that in one embodiment integrates directly with the Nett Warrior network to deliver medical countermeasures (MCMs) such as nalmefene and atropine/ipralidoxime chloride in a single ruggedized device.

Survivability on the battlefield where chemical agents are potentially present requires fast delivery, with connected, coordinated response across the force. The system delivers the appropriate antidote for either nerve agent or opioid aerosol exposure. The system interacts with the warrior so that drug is only delivered when required without the need for self or rescuer administration. It communicates with the Nett Warrior system to provide network-aware capability. In an alternative embodiment, the system delivers multiple drugs on the battlefield and is not limited to only one or two.

There is thus provided in accordance with the invention, a method of detecting and treating hazardous substance exposure of a warfighter for use in a wearable countermeasure system, the method comprising rendering a plurality of interactive challenges to the warfighter, continuing to present challenges to the warfighter as long as the warfighter successfully passes said challenges, upon failing a challenge, applying a stimulus to the warfighter and continuing to present said challenges, and upon failing a predetermined number of challenges, initiating injection of one or more bioactive substances to the warfighter.

There is also provided in accordance with the invention, a method of detecting and treating hazardous substance exposure of a warfighter for use in a wearable countermeasure system, the method comprising receiving an alert indicating exposure or potential exposure to hazardous material, in response to the alert, rendering a plurality of interactive challenges to the warfighter, continuing to present challenges to the warfighter as long as the warfighter successfully passes said challenges, upon failing a challenge, applying a stimulus to the warfighter and continuing to present said challenges, and upon failing a predetermined number of challenges, initiating injection of one or more bioactive substances to the warfighter.

There is further provided in accordance with the invention, a wearable countermeasure system for detecting and treating hazardous substance exposure of a warfighter, comprising a housing, a user interface for accepting one or more user commands and displaying information, an injector assembly operative to inject the warfighter with one or more bioactive substances in response to an inject command, a stimulus unit operative to generate a stimulus to be applied to the warfighter, a controller comprising instructions stored in a memory and operative to receive an alert indicating exposure or potential exposure to a hazardous substance, in response to the alert, render a plurality of interactive challenges to the warfighter, continue to present challenges to the warfighter as long as the warfighter successfully passes said challenges, upon failing a challenge, apply a stimulus to the warfighter and continue to present said challenges, and upon failing a predetermined number of challenges, generate the inject command to inject one or more bioactive substances to the warfighter.

### BRIEF DESCRIPTION OF THE DRAWINGS

The present invention is explained in further detail in the following exemplary embodiments and with reference to the figures, where identical or similar elements may be partly indicated by the same or similar reference numerals, and the features of various exemplary embodiments being combinable. It should be expressly understood that the drawings are included for illustrative purposes and do not represent the scope of the present system. It is to be understood that the figures may not be drawn to scale. Further, the relation between objects in a figure may not be to scale and may have a reverse relationship as to size. The figures are intended to bring understanding and clarity to the structure of each object shown, and thus, some features may be exaggerated in order to illustrate a specific feature of a structure. In the accompanying drawings, like reference numbers in different drawings may designate identical or similar elements, portions of similar elements and/or elements with similar functionality. The present system is explained in further detail, and by way of example, with reference to the accompanying drawings which show features of various exemplary embodiments that may be combinable and/or severable wherein:

FIG. 1 is a diagram illustrating the front portion of an example wearable drug delivery device constructed in accordance with the present invention;

FIG. 2 is a diagram illustrating the rear portion of an example wearable drug delivery device constructed in accordance with the present invention;

FIG. 3 is a diagram illustrating an example incapacitated warfighter with the wearable drug delivery device in action responding to an active CBRN threat;

FIG. 4 is a diagram illustrating an example multi-route countermeasure delivery platform Nett Warrior integrated wearable countermeasure system;

FIG. 5 is a block diagram illustrating an example wearable drug delivery device of a multi-route countermeasure delivery system;

FIG. 6 is a block diagram illustrating a mobile phone, tablet, or other portable digital device of a multi-route countermeasure delivery system in more detail including example applications executing thereon;

FIG. 7 is a block diagram illustrating an example wearable drug delivery device of a multi-route countermeasure delivery system in more detail including example applications executing thereon;

FIG. 8 is a block diagram illustrating an example cloud server of a multi-route countermeasure delivery system in more detail including example applications executing thereon and databases maintained therein;

FIG. 9 is a flow diagram illustrating example distress detection and antidote delivery utilizing a challenge with injection delay without hazardous substance detection;

FIG. 10 is a flow diagram illustrating example distress detection and antidote delivery utilizing a challenge with injection delay with personal hazardous substance detection;

FIG. 11 is a flow diagram illustrating example distress detection and antidote delivery utilizing a challenge with injection delay with external human hazardous substance alert; and

FIG. 12 is a flow diagram illustrating example distress detection and antidote delivery utilizing a challenge with injection delay with external machine hazardous substance monitoring and alert.

### DETAILED DESCRIPTION

In the following detailed description, numerous specific details are set forth in order to provide a thorough understanding of the invention. It will be understood by those skilled in the art, however, that the present invention may be practiced without these specific details. In other instances, well-known methods, procedures, and components have not been described in detail so as not to obscure the present invention.

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary, for the understanding and appreciation of the underlying concepts of the present invention and in order not to obfuscate or distract from the teachings of the present invention.

Any reference in the specification to a method should be applied mutatis mutandis to a system capable of executing the method. Any reference in the specification to a system should be applied mutatis mutandis to a method that may be executed by the system.

Throughout the specification and claims, the following terms take the meanings explicitly associated herein, unless the context clearly dictates otherwise. The phrases “in one embodiment,” “in an example embodiment,” and “in some embodiments” as used herein do not necessarily refer to the same embodiment(s), though it may. Furthermore, the phrases “in another embodiment,” “in an alternative embodiment,” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The following definitions apply throughout this document.

The term warfighter is defined as is a member of the armed forces, i.e. a soldier, sailor, airman, or marine, actively engaged in combat, deployed to a conflict zone, or authorized to use military force. A warfighter is a person responsible for making decisions involving the use of military force and directly engaged in or supporting combat operations. The term ‘user’ is also intended to refer to the warfighter and both terms are intended to refer to the same thing.

The term medical officer is used to refer to any medical related role in the military such as, but not limited to, combat medic, corpsman, commander, officer, medical technician, medical warrant officer, commissioned medical officer, medical service corps, special ops medical personnel, and biomedical officer.

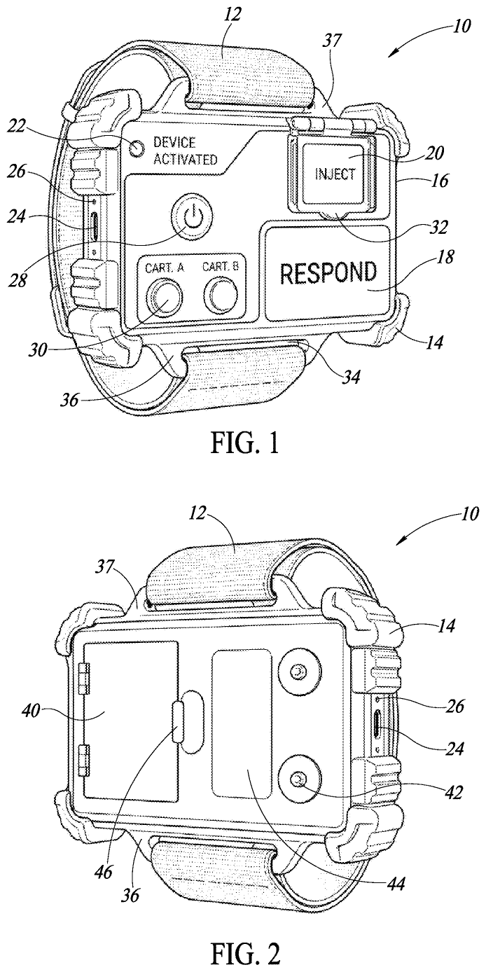

A diagram illustrating the front portion of an example wearable drug delivery device constructed in accordance with the present invention is shown in FIG. 1. A diagram illustrating the rear portion of an example wearable drug delivery device constructed in accordance with the present invention is shown in FIG. 2. Referring to FIGS. 1 and 2, a wearable drug delivery device, generally referenced 10, is illustrated in front and rear perspective views, respectively, and comprises a housing 16, wrist/arm strap 12, corner protectors 14, respond button 18, inject button 20, device activated indicator 22, charging port 24, speaker holes 26, on/off power switch 28, cartridge select buttons 30 (e.g., Cart. A and Cart. B), inject button protective cover 32, wrist strap slit opening 34, wrist strap support 36, drug injector cartridge door 40 and latch 46, electrical stimulus electrodes 42, and battery access cover 44.

In one embodiment, the device 10 is configured for rapid, single-handed drug/antidote (i.e. medicament) delivery in austere or tactical environments, including but not limited to military field operations, emergency medical response, and combat casualty care. The device is operative to be operable by the wearer or by a first responder, including under conditions of limited visibility, physical stress, or while wearing protective gloves.

The housing 16 forms the primary structural body of the device and is configured to enclose and protect the internal medicament delivery components. In one embodiment, the housing is formed from a high-impact polymer, such as glass-filled nylon, polycarbonate, or a fiber-reinforced thermoplastic elastomer, selected for its combination of structural rigidity, low weight, and resistance to deformation under mechanical load. In alternative embodiments, the housing may be formed from a metallic material such as anodized aluminum or titanium alloy, or a combination of metallic and polymeric materials, to further enhance structural integrity in high-stress environments.

The exterior surfaces of the housing are configured to withstand exposure to moisture, dust, extreme temperatures, and mechanical impact, and may be rated to applicable military standards including MIL-STD-810 for environmental durability and MIL-STD-461 for electromagnetic compatibility. In a preferred embodiment, all seams, ports, and apertures of the housing are sealed with resilient gaskets or O-rings formed from a fluoroelastomer or silicone-based material to provide ingress protection rated to at least IP67 under IEC 60529, thereby rendering the device submersible and resistant to contamination by biological or chemical agents.

The housing further comprises a plurality of corner modules 14 disposed on all four corners of the housing. The corner modules are formed from an overmolded elastomeric material and serve as impact bumpers, providing shock absorption in the event of a drop or impact. The housing may additionally house one or more electronic subassemblies, antenna elements, or auxiliary control interfaces. A charging and data port 24 is recessed within a lateral face of the housing and optionally sealed by a removable port cover (not shown), the port being configured to accept a standardized electrical connector such as USB-C or a proprietary waterproof connector for recharging an internal power source and for bidirectional data communication with an external computing device.

The strap assembly 12 is configured to releasably secure the device to the wrist or arm of a user and is constructed to remain stable under conditions of vigorous physical activity. In a preferred embodiment, the strap assembly comprises a woven textile substrate formed from a high-tensile-strength material such as nylon webbing, MOLLE-compatible ballistic nylon, or an aramid fiber weave, providing tensile strength sufficient to prevent unintended detachment of the device during field operations.

The strap assembly 12 is coupled to the housing at an upper strap anchor 37 disposed at the proximal end of the housing and at a lower strap anchor 36 disposed at the distal end, such that the housing is suspended between the two anchor points and oriented with its front-facing control panel directed away from the skin surface of the user. In one embodiment, the strap assembly incorporates a quick-release buckle mechanism configured to allow single-handed donning and doffing of the device. In one embodiment, the strap assembly may be configured for attachment to a MOLLE vest, plate carrier, or other tactical load-bearing equipment, enabling the device to be worn on locations other than the wrist, including the forearm, upper arm, or thigh. In this case, however, the device will not be able to inject drug(s) into the warfighter as it is not on the wrist or arm.

The rear of the housing assembly may further incorporate a biometric sensor interface on its inner surface, configured to contact the skin of the user and to detect physiological parameters including but not limited to heart rate, blood oxygen saturation, skin temperature, and galvanic skin response, thereby enabling the device to monitor the physiological state of the user and to condition or modify drug delivery based on detected parameters.

The device is configured to house a plurality of drug (i.e. medicament) cartridges within an internal cartridge bay accessible via the rear access panel 40. In an embodiment, the device accommodates at least two discrete cartridges, designated Cartridge A and Cartridge B, each independently selectable via a corresponding cartridge selector button 30 disposed on the front-facing control panel. It is appreciated that the device is not limited to two drug cartridges and can accommodate additional drug cartridges depending on the actual implementation (e.g., four, six, or more). In addition, not all cartridges necessarily contain only chemical warfare related drugs and antidotes, but rather can contain any type of bioactive substances such as nutritional and metabolic support compounds (vitamins, electrolytes, glucose, etc.), performance enhancing compounds (e.g., caffeine, modafnil, creatine, beta-alanine, etc.), stress and cognitive support compounds (e.g., stress and cognitive support compounds (e.g., adaptogens, magnesium glycinate, etc.), and immune and recovery support compounds (e.g., glutathione, zinc, collagen peptides, etc.).

In general, the cartridges can contain any type of bioactive substance, including but not limited to pharmacological agents, drugs, chemical warfare agent antidotes, opioid antidotes, nutritional supplements, vitamins, mineral, electrolytes, adaptogens, hormone precursors, performance-enhancing compounds, diagnostic agents, or any combination thereof.

Each cartridge is a self-contained, pre-filled, sterile medicament reservoir configured for single or multiple-dose delivery. The cartridges may contain any of a variety of bioactive substances, e.g., pharmacological agents, appropriate to the intended operational context, including but not limited to analgesics such as morphine or ketamine, hemostatic agents, antidotes to chemical or biological agents such as atropine or pralidoxime, vasopressors, antibiotics, or sedatives. In the example embodiment presented herein, the dual-cartridge architecture permits the storage and selective delivery of two distinct bioactive substances including medicaments within a single wearable platform, enabling sequential or condition-dependent administration without requiring the user to handle or load additional equipment in the field. As described supra, the device may be designed to hold a larger number of bioactive substances.

Each cartridge selector 30 comprises a rotary dial or depressible selector button or switch disposed on the front control panel, the actuation of which fluidically couples the selected cartridge to the injection assembly via an internal valve manifold. In one embodiment, the valve manifold is a microfluidic assembly comprising one or more solenoid-actuated valves configured to establish a fluid pathway from the selected cartridge to the injection needle or delivery port upon receipt of an electrical actuation signal. The valve manifold is further configured to prevent cross-contamination between cartridges by maintaining discrete fluid pathways for each cartridge.

The cartridge bay is accessed via the rear access panel (door) 40, which is hingedly mounted to the housing and secured in a closed position by one or more retention fasteners (not shown). A latch or release mechanism 46 is provided to selectively disengage the retention fasteners and permit opening of the access panel for cartridge replacement or inspection. In one embodiment, the cartridge bay is configured to accept cartridges in a standardized form factor, and the cartridges are keyed or color-coded to prevent incorrect insertion.

The inject button 20 is disposed within the housing and comprises a protective cover 32. When pressed it activates an internal drug delivery mechanism. The protective cover 32 is hingedly or slidably mounted to the housing and is biased toward a closed position by a resilient member such as a spring or elastomeric detent, thereby preventing inadvertent actuation of the inject button during normal wear. In one embodiment, the protective cover 32 must be deliberately displaced, i.e. by lifting, sliding, or rotating, prior to actuation of the inject button, providing a two-step safety interlock against accidental drug delivery.

Upon displacement of the protective cover 32 and depression of the inject button, the device immediately initiates a drug delivery sequence. In one embodiment, the internal delivery mechanism comprises an auto-injector assembly including a drive spring, a plunger, and a needle assembly configured to extend from a skin-contacting surface of the housing or strap assembly upon actuation. The needle assembly is retractable and configured to return to a retracted, shielded position following completion of the injection cycle, thereby minimizing the risk of needlestick injury. In an alternative embodiment, the delivery mechanism may comprise a needle-free injection system utilizing a high-pressure gas cartridge or piezoelectric actuator to transdermally deliver the medicament without percutaneous needle penetration.

The drug delivery process may be controlled by an internal microcontroller configured to regulate the volume and rate of medicament delivery, to log the time and identity of each delivery event, and to prevent re-actuation within a defined lockout period following a completed injection, thereby reducing the risk of overdose. Delivery parameters including dosage volume, delivery rate, and lockout interval may be pre-programmed at the time of manufacture or configured by authorized personnel via the data port 24.

The front-facing control panel of the device presents a simplified, tactile user interface configured for reliable operation under stress, in darkness, rugged conditions, or while wearing gloves. The control panel comprises a status indicator 22, a power actuator 28, a response actuator 18 labeled “RESPOND,” a first cartridge selector (Cart. A) and a second cartridge selector (Cart. B).

The status indicator 22 comprises a visual indicator, preferably an illuminated LED element, configured to display the operational state of the device, i.e. that it is activated. In one embodiment, the status indicator 22 displays a first color, such as green, when the device is armed and operational, a second color, such as red, when a fault condition is detected, and no illumination when the device is in a standby, inactivated, powered-down state. The status indicator 22 may additionally provide audible or haptic feedback via an integrated speaker or vibration motor.

In one embodiment, the power actuator 28 is a recessed push-button switch configured to power the device on or off. In one embodiment, the power actuator 28 requires a sustained depression of at least two seconds to toggle the power state, preventing inadvertent activation or deactivation.

The response actuator 18, labeled “RESPOND,” is a large-format, prominently positioned push-button configured for rapid actuation with minimal precision. The main use of this button is by the warfighter to respond during challenges presented by the device. Alternatively, or in addition, upon actuation, the response button transmits a distress signal or status notification via an integrated wireless communications module to a remote monitoring station, commanding officer's device, medical officer's device, or networked medical personnel. The wireless communications module may operate over one or more radio frequency protocols including but not limited to Bluetooth Low Energy, ANT+, LoRa, or a military-grade encrypted RF channel, Nett Warrior system, etc.

The operational sequence for drug delivery proceeds as follows: the warfighter powers on the device and confirms that the device is in an activated state as indicated by the status indicator 22; selects the desired cartridge via the appropriate cartridge selector 30 or receives a command whereby the device automatically selects one of the cartridges for potential injection. To initiate self-injection, the warfighter user displaces the protective cover 32; and depresses the inject button 20 to initiate drug delivery. The device confirms successful delivery via the status indicator 22 and, in one embodiment, transmits a delivery event log entry wirelessly to a connected monitoring system. The response actuator 18 may subsequently be actuated in response to a post injection physical stimulus and/or to notify remote personnel of the medical event.

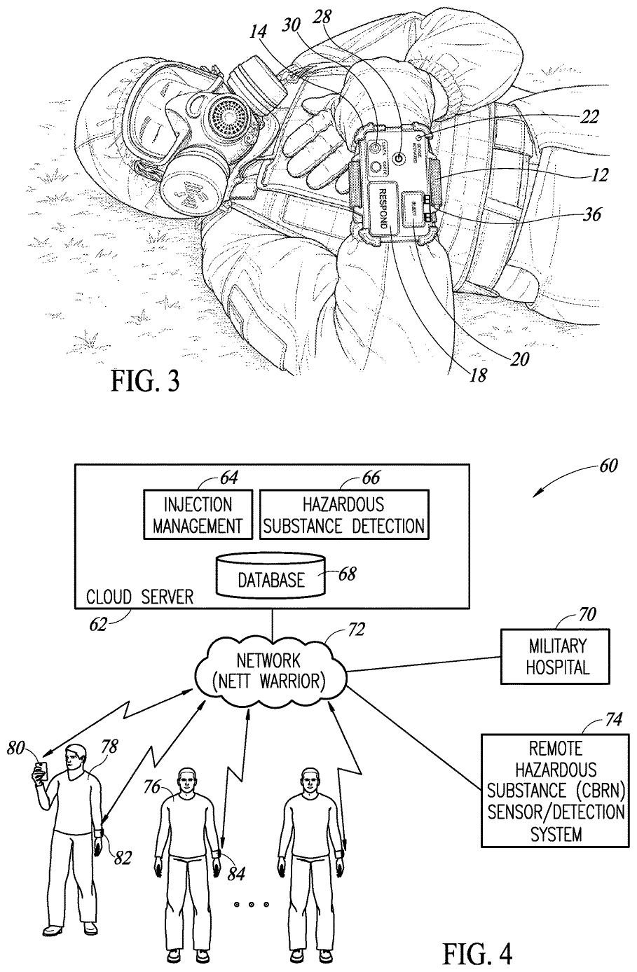

A diagram illustrating the wearable drug delivery device in an operational context, depicting a user in a incapacitated state receiving autonomous delivery of a bioactive substance via the device while equipped with full chemical, biological, radiological, and nuclear (CBRN) protective equipment is shown in FIG. 3. The wearable drug delivery device is shown deployed in a representative operational scenario. A user, depicted as a military warfighter, is shown in a supine position on a ground surface, in a state of incapacitation due to exposure to a chemical, biological, radiological, or nuclear (CBRN) threat agent. The user is equipped with a full-face respirator mask and protective outer garments that are related to CBRN personal protective equipment (PPE), illustrating the device's intended operational environment and the conditions under which it may be required to function.

In this example application, the device is worn on the wrist of the user and is visible on the user's forearm, secured by the strap assembly. Note that although a single warfighter is shown, a fellow warfighter or first responder may manipulate the device as well such as with a gloved hand, demonstrating that the device is configured for operation both by the wearer and by assisting personnel, and that actuation of the device is achievable while wearing standard military protective gloves without requiring fine motor dexterity or the removal of protective equipment.

The front-facing control panel of the housing is visible with the status indicator 22 visible in an activated state, indicating that the device has detected or been remotely or manually armed in response to a threat condition. The inject button 20 including the protective cover 32 and respond button 18 are shown in an accessible configuration. The challenge response button 18, labeled “RESPOND,” is prominently visible on the lower portion of the control panel. The cartridge selectors 30 are visible in the lower left portion of the control panel, with the corner protectors 14, strap 12, strap supports 36, device activated indicator, and power button 28 also shown.

This illustrates a primary use case of the device in which a warfighter who has been rendered incapacitated, whether by exposure to a nerve agent, blister agent, blood agent, or other CBRN threat, is unable to self-administer treatment. In this scenario, the device determines that the warfighter, via a sequence of challenges, is incapacitated and in response initiates injection of the warfighter with an antidote. It is also possible that a fellow warfighter or combat medic actuates the device on behalf of the incapacitated user without requiring removal of either party's protective equipment, without requiring access to a medical kit or separately carried auto-injector, and without requiring the assisting individual to make direct skin contact with the incapacitated user or to breach the user's protective garments.

In one embodiment, the device is pre-loaded with at least one cartridge containing an antidote appropriate to the anticipated CBRN threat environment. In the context of nerve agent exposure, including but not limited to organophosphate compounds such as sarin (GB), soman (GD), tabun (GA), VX, or Novichok-class agents, the cartridge may contain atropine sulfate, pralidoxime chloride (2-PAM CI), diazepam, or a combination thereof, consistent with current military treatment protocols for nerve agent casualties such as those embodied in the CHEMPACK program and the Mark 1 antidote kit. In the context of cyanide or blood agent exposure, the cartridge may contain hydroxocobalamin or sodium thiosulfate. The dual-cartridge architecture of the device, comprising Cartridge A and Cartridge B selectors, enables the pre-loading of two distinct antidotes within the single wearable platform, permitting sequential administration of a two-component antidote regimen, such as atropine followed by pralidoxime, without requiring the assisting warfighter to locate, prepare, or handle separate injection devices.

In another embodiment, the device is configured to detect exposure to a CBRN threat agent autonomously, via one or more integrated chemical sensors, biosensors, or physiological monitors disposed within or on the housing or strap assembly, and to initiate an automated drug delivery sequence upon detection of predetermined physiological or environmental trigger conditions, without requiring manual actuation by the user or an assisting individual. In addition, the device may receive an alert from an external human or machine source indicating exposure or potential exposure to a hazardous substance.

Such trigger conditions may include, without limitation, a detected concentration of a nerve agent or other toxic compound in the ambient environment exceeding a threshold level, a detected change in the user's physiological parameters consistent with toxic agent exposure such as bradycardia, miosis, seizure activity, or loss of consciousness determined via repeated challenge failures, or a combination of environmental and physiological indicators. This autonomous delivery capability is of particular significance in scenarios such as that depicted in FIG. 3, wherein the user may be rendered immediately and completely incapacitated by agent exposure and therefore unable to initiate self-treatment.

The scenario depicted in FIG. 3 further illustrates the advantage of the wrist-worn form factor of the device over conventional auto-injector systems such as the military-issue ATNAA (Antidote Treatment Nerve Agent Auto-Injector) or DuoDote injector, which require the assisting individual to locate the injector within the casualty's gear, remove protective caps, and manually inject into exposed skin, steps that are difficult or impossible to perform while wearing CBRN protective gloves and that may require partial removal of the casualty's protective garments, potentially exposing both the casualty and the responder to additional agent contamination. The device eliminates these steps by providing a fixed, wrist-worn, immediately accessible delivery platform operable through a gloved interface, thereby reducing time-to-treatment and minimizing responder exposure risk.

A system architecture diagram illustrating an example multi-route countermeasure delivery platform Nett Warrior integrated wearable countermeasure system is shown in FIG. 4. The system, generally referenced 60, comprises a plurality of warfighters 76, each outfitted with a networked wearable drug delivery device 84, a medical officer or unit commander 78 with the wearable device 82 and a connected phone/tablet 80, a remote hazardous substance (CBRN) sensor and detection system 74, a cloud-based server infrastructure 62, and a military hospital or higher echelon medical facility 70.

The networked drug delivery and hazardous substance detection system 60 is configured to integrate the wearable drug delivery device 84 worn by individual warfighters with a broader tactical network infrastructure, enabling real-time monitoring of warfighter physiological status and CBRN exposure events, coordinated command and control of drug delivery operations across a plurality of devices, automated hazardous substance detection and alert dissemination, and transmission of medical event data to higher echelon medical facilities for continuity of care.

At the apex of the system 60 is a cloud server 62, comprising a distributed computing infrastructure configured to receive, process, store, and transmit data from and to all nodes within the system 60. The cloud server 62 hosts at least two principal software modules: an injection management module 64 and a hazardous substance detection module 66, and maintains one or more databases 68 for persistent storage of operational and medical data.

The injection management module is configured to receive, log, and process injection event data transmitted from each wearable drug delivery device within the network. Such data may include, without limitation, the identity of the warfighter, the timestamp of the delivery event, the identity and quantity of the bioactive substance delivered, the cartridge from which delivery was made, the physiological parameters of the warfighter at the time of delivery, and the GPS coordinates of the delivery event. The injection management module 64 is further configured to transmit alerts, delivery confirmations, and treatment recommendations to authorized personnel within the network, including the medical officer or commander 78 and connected medical facilities 70. In one embodiment, the injection management module is configured to enforce dosing protocols and lockout intervals across networked devices, preventing overdose in scenarios where multiple responders may attempt to actuate the device of an incapacitated warfighter in rapid succession.

The hazardous substance detection module 66 is configured to aggregate and analyze hazardous substance detection data received from a plurality of sources within the network, including the integrated sensors of individual wearable devices and the remote hazardous substance sensor and detection system. The module 66 applies threat assessment algorithms to the aggregated sensor data to determine the nature, concentration, and spatial distribution of detected threat agents, and to generate threat alerts and recommended countermeasures for dissemination to connected network nodes. In a preferred embodiment, the module 66 is configured to correlate environmental sensor data with warfighter physiological data to identify exposure events and to trigger or recommend autonomous drug delivery sequences on affected devices.

The plurality of databases 68 comprise one or more persistent data stores hosted on the cloud server 62 and configured to maintain records including warfighter medical profiles, pre-programmed delivery protocols, cartridge inventory and expiration data, historical injection logs, sensor event logs, and threat intelligence data. The databases are accessible to authorized personnel including medical officers, commanders, and medical facility personnel via the network 72, subject to appropriate access controls and encryption.

The cloud server 62 communicates with all nodes of the system 60 via a tactical network 72, depicted in FIG. 4 as a network cloud element. In one embodiment, the network comprises the NETT Warrior network, a US Army fielded command and control system providing situational awareness and digital communications at the dismounted soldier level. In an alternative embodiment, the network may comprise any suitable military or commercial wireless communications infrastructure, including but not limited to tactical radio networks operating on encrypted RF channels, satellite communications networks, mesh radio networks, cellular LTE or 5G networks, or a hybrid combination thereof. The network is configured to provide bidirectional data communications between the cloud server 62 and each connected node, including the wearable devices, the medical officer or commander's phone/tablet device 80, and the remote hazardous substance sensor and detection system 74.

A plurality of warfighters 76 are each equipped with a wearable drug delivery device 84 secured to the wrist or body as described supra. Each device worn by a warfighter constitutes a network node and is assigned a unique device identifier within the system. Each device is configured to transmit physiological sensor data, CBRN exposure detection data, injection event data, and device status data to the cloud server 62 via the network 72 on a continuous or event-triggered basis. Each device is further configured to receive alerts, delivery instructions, and updated threat protocols from the cloud server or from the medical officer or commander's device 80 via the network.

It is noted that the system 60 is scalable of accommodating any number of networked devices, from a small fireteam to a company or battalion sized element, for example. Each device within the network operates independently in the event of loss of network connectivity, reverting to its onboard autonomous delivery protocols as described herein, thereby ensuring continuity of protection for the individual warfighter regardless of network availability.

A medical officer or unit commander 78 is depicted as a network node connected to the tactical network 72 via a personal computing device 80, which may comprise a smartphone, ruggedized tablet, wearable display, or other portable computing device configured with a command and control application interfacing with the injection management module 64 and hazardous substance detection module 66 of the cloud server 62. Via the device 80, the medical officer or commander 78 is able to monitor in real time the status, location, physiological parameters, and injection history of each warfighter 76 within the network; receive immediate alerts upon detection of a CBRN exposure event or injection actuation; remotely authorize, generate and transmit alerts, initiate, or override drug delivery sequences on individual or multiple devices; adjust delivery protocols or cartridge selection parameters across the network; and transmit medical event reports to the military hospital 70 or higher echelon medical command.

A remote hazardous substance (CBRN) sensor and detection system 74 is depicted as a network node connected to the tactical network 72 and to the cloud server 62. The system 74 comprises one or more fixed, vehicle-mounted, aerial, or unattended ground sensor platforms configured to detect the presence and concentration of hazardous substances in the operational environment, including but not limited to nerve agents, blister agents, blood agents, choking agents, toxic industrial chemicals, radiological materials, and biological threat agents. Detection data from the system 74 is transmitted in real time to the hazardous substance detection module 66 of the cloud server 62, where it is correlated with warfighter location data to identify individuals within a detected threat plume or contamination zone and to trigger appropriate alerts and autonomous delivery sequences on the corresponding wearable devices.

In one embodiment, the remote sensor system 74 comprises a networked array of chemical agent detectors such as ion mobility spectrometry (IMS) sensors, photoionization detectors (PID), surface acoustic wave (SAW) sensors, or mass spectrometry-based detection systems, optionally supplemented by meteorological sensors providing wind speed and direction data to enable predictive modeling of threat agent dispersion and advance warning to warfighters in the projected path of a threat plume.

In one embodiment, the remote sensor system 74 may comprise (1) stand-off (remote) sensor capable of detecting hazards from a distance including infrared spectroscopy (FTIR), laser based systems (LIDAR/DIAL), and UV/IR imaging; (2) point detectors (on robots or fixed sites) including ion mobility spectrometry (IMS), mass spectrometry/gas chromatography (GC-MS), electrochemical sensors, and photoionization detectors (PID); (3) biological detection including PCR based systems (DNA amplification), immunoassays, and fluorescence; (4) platforms including unmanned ground vehicles (UGVs), unmanned aerial vehicles (UAVs/drones), and fixed/networked sensors. Examples include joint chemical agent detector (JCAD), Stryker NBCRV, RAID system, and M4 JCAD.

A military hospital or higher echelon medical facility 70 is depicted as a network node connected directly to the cloud server 62, outside the tactical network 72, representing a reach-back medical capability. The facility 70 receives real-time and historical medical data from the cloud server 62 pertaining to CBRN exposure events and drug delivery actions occurring in the field, enabling medical personnel at the facility 70 to prepare for the reception of casualties, to advise field medical officers on treatment protocols, and to maintain a continuous chain of medical custody from point of injury through definitive care. In one embodiment, the facility 70 is connected via a secure, encrypted wide-area network link to the cloud server 62, and is provided with a dedicated medical dashboard application configured to display the medical status and treatment history of all warfighters within the system 60.

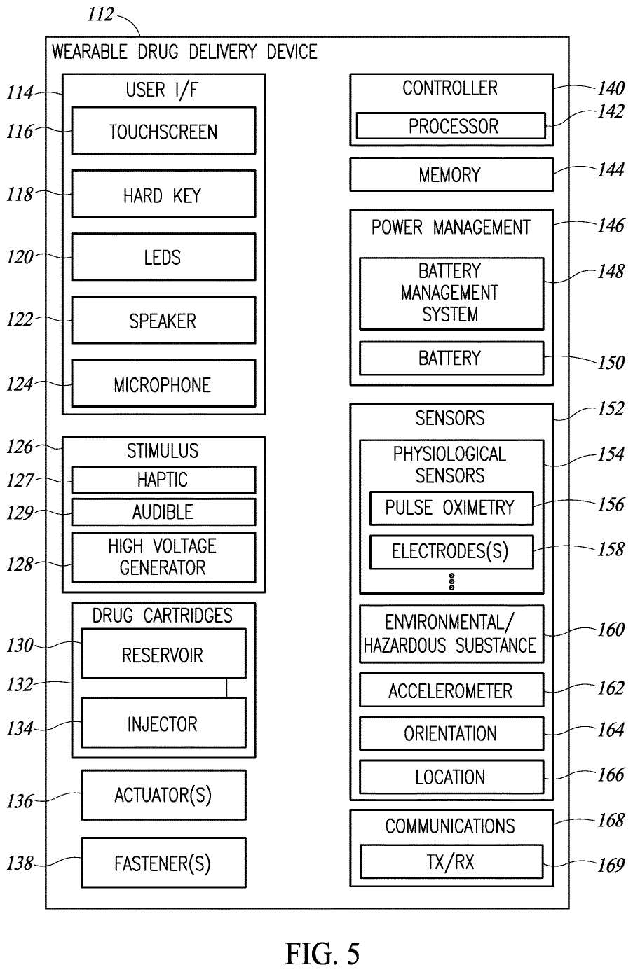

A block diagram illustrating an example wearable drug delivery device of a multi-route countermeasure delivery system is shown in FIG. 5. The wearable drug delivery device, generally referenced 112, is a key component of the remote hazardous substance (CBRN) sensor and detection system in accordance with embodiments of the present system. In one embodiment, the device includes one or more of a user interface (I/F) 114, stimulus unit 126, drug cartridges 132, controller 140, memory 144, power management unit 146, sensors 152, fasteners 138, actuators 136, and communications block 168, one or more of which may communicate with the other using any suitable communication method or methods such as wired (e.g., using a local bus, etc.), wireless, and/or optical communication methods over the network 72 (FIG. 4) and may be coupled to at least one of a body or housing of the wearable.

The user I/F 114 comprises a touchscreen, hard keys, LEDs, speaker, and microphone. The stimulus 126 comprises haptic engine 127, audible generator 129, and electric stimulator (e.g., high voltage generator and shocker). The drug cartridges 130 comprises reservoir 132 and injector mechanism 134. The power management 146 comprises battery management system 148 and battery 150. The sensors 152 comprises physiological sensors 154 including pulse oximetry 156, electrodes 158, etc., environmental/hazardous substance 160, accelerometer 162, orientation 164, and location 166. The communications 168 comprises Tx/Rx unit 169.

The body may define at least a portion of at least one cavity configured to receive one or more of the stimulus unit, drug cartridges, controller, memory, power management unit, sensors, fastener, and communications block. The body may be secured to a desired area of the warfighter by the fasteners which may be passive and/or active. The fasteners may include a band that may be formed integrally with or separately from the body. The fasteners may include a motor (e.g., linear or rotary controlled by the controller), buckle, rubber band, elastomeric band, hook and loop fastener (e.g., Velcro™, etc.), threaded coupling, snap-fit coupling, and/or any suitable coupler. The fastener may include a sensor to sense when it is secured to the arm, form a signal indicating such, and transmit this signal to the controller for further analysis such as to determine when the fastener is sufficiently secured to the warfighter.

The body may include one or more openings extending therethrough and configured to receive one or more of the drug cartridges. A coupler may be provided to releasably couple one or more of the injector and the reservoir to the body. In some embodiments, an injection cartridge (not shown) may include a body including the injector and the reservoir and may be releasably coupled to the body of the wearable using any suitable coupler such as a threaded fastener, a latch, a snap fit joint, a bayonet-type fastener, an interference fit, etc. The injection cartridge may be coupled to one or more of the actuators to control an injection of a bioactive substance under the control of the controller.

The fastener may be coupled to the body and may be configured to position the body relative to the warfighter during use. The fastener may include an adjuster such as a hook and loop fastener (e.g., VELCRO™ or the like) configured to adjust a length and/or tension of the fastener. The fastener may include an adhesive strip or gel may be applied to an underside of the body which may be configured to releasably couple the body of the wearable to the warfighter. This gel may include a conductive medical grade gel and may be coupled to at least one electrode.

The memory may include any suitable memory or memories that may store operating instructions, algorithms, models, SSI, QUID, health records (e.g., EHRs, EMRs, OURs, or portions thereof), SI, and/or information generated by the wearable for later use. The memory 144 may store information used, generated, acquired, and/or updated by the wearable and may be local and/or distributed throughout the system.

The controller may control the overall operation of the wearable and may include one or more logic devices such as a microprocessor 142 and/or the like which may be local and/or distributed throughout the wearable. The controller may be configured to execute instructions such as operating instructions which may be stored in a memory of the system such as the memory or other suitable memory and may perform arithmetic operations in accordance with embodiments of the present system and may include digital and/or analog circuitry. The microprocessor may control the overall operation of the controller and may include one or more of arithmetic, logic, and/or control circuitry configured to perform one or more functions of a central processing unit (CPU). The microprocessor may include one or more processors such as a general purpose processor including a plurality of functions at least one of which may be operative in accordance with embodiments of the present system. In some embodiments, the microprocessor may include a graphics processing unit (GPU), a digital signal processor, a neural processing unit (NPU), and/or the like.

The power management portion may include one or more of a battery management system (BMS) 148 and a power source such as a battery 150 (or capacitor or other power storage device) and may be configured to provide power to one or more portions of the wearable such that the wearable may be operative as a standalone unit with its own power. For the sake of clarity, the power source will be assumed to include only the battery in the present embodiments although other energy storage devices such as capacitors are also envisioned. It is also envisioned that the battery may include a plurality of batteries that may be local or distributed relative to each other. In some embodiments, the power management portion may manage power to one or more portions of the wearable in accordance with an operating mode. For example, the wearable may have on, off, sleep, low power, and operating modes in which power may be provided to portions of the system in accordance with system settings. In the low power mode, power may be provided only to select portions of the system as may be set forth in system settings. In the off mode power may be completely off or may be provided to certain portions of the system such as an on/off switch (if required) and/or one or more sensors as may be set forth in system settings.

The power management portion may include a charging port configured to receive power from an external source suitable for charging the battery. The charging port may include any suitable type of port such as a USB port (e.g., USB-C, micro USB, etc.), Lightning port, Thunderbolt port, wireless port, magnetic charging port (e.g., MagSafe or other inductive charger), and/or proprietary port and may be located at or near a periphery of the body.

The power management portion may include one more sensors configured to monitor one or more of voltage, current, and temperature (Tbatt) of the battery, form corresponding information, and provide this information to the controller and/or BMS. The BMS may be configured to monitor health, state of charge, number of cycles, voltage, current, charge, and balance, of the battery and may be configured control power to and/or from the battery accordingly. In some embodiments, a heater may be provided to warm the battery to operating temperatures. For example, the controller may determine whether Tbatt is less than a threshold operating temperature (Top). If it is determined that Tbatt is less than Top, the controller may be operative to turn on the heater to warm the battery. If it is determined that Tbatt is not less than Top, the controller may be operative to turn off the heater. This may be beneficial when operating in outdoor or other environments where power storage of the battery may be adversely affected below certain temperatures. The controller may render an indication of battery temperature for the convenience of the warfighter and may provide an interface with which to adjust battery heater settings (e.g., on, off, etc.) which settings may be stored in the SSI. In some embodiments, portions of one or more processes may not start until temperatures of one or more temperature monitored portions of the system are within an operating temperature range or ranges. Heaters may be provided to warm certain portions of the system when it is determined that a corresponding portion of the system is below a threshold temperature.

The BMS may provide information relating to charge of the battery to the controller for further analysis and/or for rendering on a rendering device of the system such as on a touchscreen 116, light emitting diodes (LEDs) 120 of the user I/F and/or on a display. The controller may be configured to render information relating to charge using one or more colors to indicate an available power level such as green for fully charged, yellow for charge soon, and red for insufficient charge (must be charged now). The controller may provide a user interface with which the warfighter may interact to view, set, and/or reset power settings and may initiate a power saving mode to reduce a load on the battery when it is determined that the charge of the battery is below a threshold value that has been determined assure proper operation of the system. The touchscreen may include one or more displays one or more of which may be located for viewing and interaction with the warfighter during use if desired.

In some embodiments, the battery may be removed from the body for replacement and/or charging in which case portions of the BMS may be separated from the body. A coupler may be provided to couple the battery to the body.

The wearable may include a communication port configured to enable communication with other portions of the system and may be combined with, or separate from, the charging port. In the present embodiments, however, it is assumed that the communication and charging ports are integrated. For example, a USB-C port, or the like, may be provided for both communication and charging via a wired connection or the like.

The communications block 168 may include a Tx/Rx circuit 169 which may be controlled by the at least one controller and may be operative to communicate with other portions of the system to receive information therefrom and/or transmit information thereto. The Tx/Rx may be configured to transmit and/or receive information from to one or more portions of the system. This information may include, for example, sensor information, operating instructions, operating status, etc. for operating in accordance with embodiments of the present system. The Tx/Rx may be configured to provide information that it receives to the controller for further processing and/or storage and may receive information from the controller for transmission to a desired receiver such as the wireless sensors and/or device of the warfighter to conserve battery power. In some embodiments, the Tx/Rx may include a low power wireless transceiver or the like and may be configured to pair to one or more portions of the system to transmit and/or receive information therefrom. The Tx/Rx may transmit in real time or may transmit in transmission bursts to conserve power. Digital signal processing of the sensor information may be performed locally or remotely from the device.

The sensors 152 may include one or more sensors such as one or more physiological sensors 154, accelerometer 162, position (i.e. orientation) sensor 164, location sensor 166, and environmental sensor 160, each of which may sense a corresponding parameter, form corresponding sensor information, and provide this sensor information to the controller for further processing and/or storage. The one or more sensors may sense information at periodic or nonperiodic intervals under the control of the controller. In some embodiments, the controller may query a sensor of the one or more sensors to provide corresponding sensor information. At least one signal conditioner may be provided to condition the sensor information prior to it being received by the controller with the conditioning being dependent upon a type of sensor information being conditioned. The one or more physiological sensors 154 may include one or more pulse oximetry sensors 156, one or more electrodes 158, respiration monitor belt (i.e. a chest belt), a respiratory sensor, and a microphone. Although other sensors are also envisioned as described supra. The one or more physiological sensors may be configured to sense corresponding physiological parameters of the warfighter, form corresponding sensor information, and/or may provide this data to the controller for further processing and/or storage. For example, the one or more electrodes 158 may couple to the skin of the warfighter and may sense electrical currents at the skin of the warfighter, form corresponding sensor information which may then be conditioned (e.g., by filtering, amplifying, rectifying, etc. as may be desired) and may then be provided to the controller for further processing. In some embodiments, the one or more electrodes may be configured to couple to a snap type adhesive electrode such as a single use snap electrode or the like which may be employed for detecting signals on the skin of the warfighter such as EMG or ECG electrodes or the like. The snap type electrode may include an adhesive layer and a conductive layer wherein the adhesive layer may be configured to adhere to the skin of the warfighter and the conductive layer may be configured to sense electrical currents on the skin of the warfighter where the electrode is attached. These electrical currents may then be conditioned (e.g., filtered, amplified, etc., by a signal conditioner) and thereafter provided to the controller for further processing. It is envisioned that the adhesive layer may secure the body of the wearable to the skin of the warfighter.

In some embodiments, the plurality of sensors may be situated apart from one another to prevent operational interference between the plurality of sensors and to assure proper operation of the sensors. In some embodiments an isolation switch may be provided to separate transmit and receive circuitry from each other to prevent interference between transmit (e.g., high voltage signals from the stimulus unit, etc.) and receive (e.g., sensed surface signals such as ECG, EMG, and/or the like) from each other. Receivers such as signal processing circuitry and the like coupled to the one or more electrodes to receive signals therefrom may be disabled to protect these receivers from damage when the stimulus unit is activated and transmits a high voltage signal waveform to the one or more electrodes. Accordingly, the system may be configured to shock the warfighter by applying multiple voltage levels (low or mild through high or intense) to the warfighter via the one or more electrodes.

The pulse oximetry sensor (pulse-ox sensor) may include at least one emitter and receiver pair which may be arranged in at least one of a transmissive or reflective pulse oximetry configurations configured to generate corresponding sensor information (e.g., pulse oximetry information) which may be processed to determine peripheral oxygen saturation (SpO2) of the warfighter and/or generate at least one photoplethysmogram (PPG) that may be transmitted to a controller of the system for further analysis and/or stored in a memory of the system for later use. The PPG may be in any suitable format such as an image format, a graph format, a streaming numerical format, etc. The controller may control the pulse oximetry sensor to sample at a sampling rate (e.g., 50 samples per second although other rates are envisioned) to obtain pulse oximetry information sufficient to form the PPG at a desired time intervals as may be set forth in the SSI. For example, PPGs may be formed every five seconds, although other latency times are also envisioned.

In some embodiments, the one or more sensors may detect the presence and/or absence of one or more of the injector 134, reservoir 130, and injection cartridge (IC) 132, form corresponding sensor information and provide this sensor information to the controller for further processing such as to determine whether the one or more of the injector, reservoir, and injection cartridge (IC), are present and secured.

The accelerometer may detect acceleration of the wearable (or a corresponding portion thereof) along, and/or about, one or more axes and form corresponding acceleration sensor information, and provide this information to the controller for further processing.

The respiration monitor belt may measure movement of the chest and/or abdominal wall of the warfighter, form corresponding sensor data, and provide this data to the controller for further processing. The controller may then measure pulmonary ventilation of the warfighter based upon this sensor data using any suitable method such as respiratory inductance plethysmography (RIP) or the like.

The orientation sensor 164 may detect orientation (e.g., relative to a magnetic field such as the Earth's magnetic field), may form corresponding orientation sensor information, and may provide this information to the controller for further processing. In some embodiments, the orientation sensor may include a magnetic field sensor such as a magnetometer or the like.

The location sensor may employ any suitable method to determine location of the device such as GPS, A-GPS, triangulation, etc., may form corresponding location sensor data, and/or may provide the location sensor data to the controller for further processing such as to determine a location of the device of the warfighter. In some embodiments (e.g., those employing A-GPS and the like) location of the device may, at least in part, be determined remotely and transmitted to the device. It is further envisioned that in some embodiments, when location information may be unavailable such as in remote and/or shielded locations, the system may estimate a current location of the device based upon acceleration and/or orientation information and a known past location of the device. This may be useful in shielded areas (e.g., large buildings, hospitals, etc.) and/or in remote locations. In some embodiments the device may provide an interface with which the warfighter may interact to enter a current location of the device and the system may use this location as a current address.

In some embodiments, the controller upon receiving one or more of the acceleration sensor information, the orientation sensor information, and/or the location sensor information may analyze the corresponding sensor information and determine acceleration, orientation, and/or location, of the device, respectively, or may transmit the corresponding sensor information to the system for further processing. This information may be useful to locate the device while in use, and/or to determine whether the device and thus the warfighter is moving (e.g., walking, running, etc.).

In some embodiments, acceleration information may be mapped to one or more inputs of the system. For example, certain motions of the device may be mapped to certain keys and/or commands such as a command to activate the device, respond to a request from the warfighter (e.g., “OK”), etc. In some embodiments, the system may determine that the warfighter is not in respiratory depression when walking motion is detected through an analysis of the acceleration information from the device. Thus, the system may monitor for movement of the device while activated and may determine that the warfighter is not suffering from respiratory distress if, for example, the warfighter is walking, running, jogging, or otherwise engaging in physical activity, etc. as may be determined through analysis of one or more of the location, acceleration, and/or orientation sensor information.

A user interface (UI) may be provided to map movements (e.g., walking, running, etc.) and/or actions (e.g., a chopping action, etc.) to corresponding inputs and the system may store this information for later use.

The user I/F 114 (UI) may comprise one or more of a display such as a touchscreen 116, one or more hard keys 118, speaker 122, microphone 124 and/or at least one LED 120 (such as solid, blinking, and/or multi-color LEDs) or other illumination sources with which the warfighter may interact with the device. In addition, voice commands can be accepted via a microphone interface. For example, the touchscreen 116, one or more hard keys 118, microphone 124, speaker 122, and at least one LED 120 may be configured to render information generated by the system for the convenience of the user under the control of the controller and the user may enter information (e.g., commands, etc.) to the system via one or more of the touchscreen, the one or more hard keys, at least one LED, and/or microphone (e.g., using voice commands, etc.).

In some embodiments it is envisioned that the one or more hard keys may be combined with a corresponding at least one LED to form a hard key that may be illuminated by the controller using one or more colors, etc. For example, the one or more hard keys may be illuminated by a corresponding one of the at least one LED under the control of the controller when it is desired to inform the user to respond and press the hard key. In some embodiments, the hard key may be mapped to a soft key which may be illuminated on the touchscreen concurrently with the hard key or in place of either key. Thus, the hard key may have a corresponding soft key or vice versa.

The reservoir may include any suitable container or assembly for containing a drug/antidote (bioactive substance) configured to counter the effect of a hazardous substance (e.g., nerve agent) exposed to the warfighter. Accordingly, the drug may temporarily reverse respiratory depression or other effects caused by the hazardous substance. The reservoir may be coupled directly, via a tube or hose, and/or a fastener (such as a Luer type fitting) to a hollow needle such as a hypodermic needle (hereinafter both of which may be referred to as a hypodermic needle unless the context indicates otherwise) configured to receive the bioactive substance. In some embodiments, the reservoir may be formed at least in part by a syringe and/or may be coupled to a syringe. In some embodiments, the reservoir may include a prefilled syringe (PFS) having a cavity including at least one bioactive substance such as a nerve agent antidote. A stopper may seal the opioid antidote into the cavity at one end and the hypodermic needle may include a cover seal that may be configured to seal the drug from passing through the hypodermic needle until pierced by the hypodermic needle. In some embodiments, a cannula may be employed in place of hypodermic needle. In some embodiments, a heater operative under the control of the controller may be provided to heat the reservoir to a desired temperature range if desired.

The actuators 136 may be configured to trigger an injector to initiate an injection when using passive injectors and to control extension and/or retraction of a hypodermic needle and/or pumping of a opioid antidote when using active injectors. In some embodiments, couplers may be provided and configured to couple the actuators to a portion of the injection cartridge to trigger the injection cartridge to inject the bioactive substance into the warfighter.

The injector may be configured to administer at least one dose of the bioactive substance from the reservoir via the hypodermic needle and may include an injection cartridge which may be disposable after use. In some embodiments depth of penetration of the hypodermic needle may be fixed or passively or actively adjustable for providing one or more of intramuscular and/or subcutaneous injection of the at least one dose from the reservoir. The injector may include one or more actuators configured to perform an injection process which may include acts of: advancing the hypodermic needle to a dispensing position (the cover seal may be pierced by the hypodermic needle at this point); administering a dose of the bioactive substance contained in the reservoir through the hypodermic needle when the hypodermic needle is in the dispensing position; and/or optionally retracting the hypodermic needle or activating a shield (e.g., a sheath) to cover the hypodermic needle after administering the dose through the hypodermic needle.

It is envisioned that the injections process may be passive or active. In some embodiments a plurality of injector such as passive and/or active injectors may be provided and may be of a single use disposable type in which one or more portions thereof may be of a single use type and disposable. The present embodiments will be assumed to include a PFS including at least one dose of a bioactive substance and a hypodermic needle coupled thereto.

Passive injectors may perform the injection process automatically when triggered by the actuator (e.g., a triggering actuator) operative under the controller. The passive injectors may include one or more preloaded actuators such as springs, pneumatic cylinders and/or combinations thereof, which when triggered may be configured to advance the hypodermic needle to a dispensing position, pump a dose of the drug through the hypodermic needle, and withdraw the hypodermic needle or extend a sheath over the hypodermic needle. The actuators may be configured to trigger the desired injector.

The active injectors may include one or more actuators (e.g., including linear and/or rotary actuators and/or pumps) operating under the control of the controller and configured to advance the hypodermic needle to the dispensing position, administer the dose of the desired drug when the hypodermic needle is determined to be in the dispensing position, and retract the hypodermic needle after administering the dose. Sensors may provide feedback to the controller via sensor data transmitted thereto continuously or at discrete points. For example, location of the hypodermic needle may be provided to the controller to indicate when the hollow needle is in a storage position and/or in the dispensing position (which may include a desired position or range of positions. The sensor data may be provided the controller as feedback information and controller may analyze the data and control one or more actuators accordingly. The actuators may include linear and/or rotary actuators and/or motors such as linear or rotary motors such as DC motors, stepper motors, piezoelectric motors, and/or the like. The position of portions of the injector such as the hypodermic needle may be determined using any suitable sensor(s) such as linear encoders, rotary encoders, mechanical switches (such as micro switches or the like), audio sensors, ultrasonic sensors, and/or the like.

In some embodiments, the injector may include an injection cartridge that may be disposable. In some embodiments, the wearable may include a plurality of injection cartridges each configured to administer a difference bioactive substance. In some embodiments, the injection cartridges may include one or more actuators which may be passive and/or active and may be disposable after use.

The stimulus unit 126, when activated, may provide an audible, haptic, chemical, mechanical, and/or electrical shock to the warfighter under the control of the controller. The chemical shock may be administered by an injector or an atomizer configured to dispense a dose of a chemical stimulant. The injector may be configured to be operative to inject the warfighter with a desired chemical stimulant.

With regard to the electrical shock, when activated, the high voltage generator 128 of the stimulus unit may generate a series of high voltage signals, each having a desired waveform and amplitude, and which may be applied to the skin of the user via a pair of electrodes to stimulate the warfighter via electric shock. Different levels of electric shock may be delivered from mild to intense depending on the circumstances. The series of high voltage signals may have a duration (Thv=2 seconds, etc.) and may have a dwell time therebetween (Tdwell=5 seconds, etc.) during which the high voltage signal may be paused. Each of the series of high voltage signals may have a waveform type, such as a sine, square, sawtooth, and triangle, with an amplitude, frequency, and duty cycle (if applicable). In some embodiments, the waveform may be changed each time it is repeated or in time. For example, in some embodiments, the amplitude and/or frequency of the waveform may be increased by a threshold amount (e.g., 10 volts, 5 Hz, respectively) each time the series of high voltage signals is repeated. This may increase the stimulation provided to the warfighter each time the electric shock is repeated. In some embodiments, the system may cycle through waveform types (e.g., from sine to square to sawtooth, etc.) each time the series of high voltage signals is repeated. The voltage, waveform, and/or duty cycle may be selected to revive the warfighter if unconscious.

When the stimulus unit is activated, the warfighter may render a selection item on the UI which when selected may be operative to stop the electrical shock (or chemical shock depending upon embodiments) applied to the warfighter via the electrodes. In some embodiments a button or key may be provided which may stop the electric shock when pressed.

In some embodiments, one or more of electrodes may be shared by two or more circuits in which case an isolator may be applied and activated to isolate the two or more circuits when the high voltage signals are generated to protect sensitive circuitry from damage due to high voltage being applied thereto. The isolator may be controlled by the controller and may be activated (e.g., toggled on) slightly before the high voltage signal is generated and deactivated (e.g., toggled off) slightly after the high voltage signal is no longer generated. It is also envisioned that acquisition and/or processing of sensor data may be suspended when the high voltage signal is applied to prevent erroneous date from being collected and processed by the system.

It is envisioned that the electrical shock may stimulate the warfighter to, for example, awaken the warfighter when unconscious and/or exposed to a hazardous substance effectively countering respiratory depression. For example, some opioid reversal antidotes like Naloxone and Nalmefene may require several minutes to effectively counter the effects of the opioid.

The fastener may be configured to hold the wearable, or portions thereof, in position during operation. The fastener may be formed integrally with or separately from, the body of the device. The fastener may include a buckle or other suitable fastener to open, close, and/or remove the device such as a hook and loop fastener (e.g., VELCRO™, etc.), a snap fit fastener, a magnetic fastener, an interference fit, an elastic band, etc., which may enable quick and effortless coupling. In some embodiments, the fastener may be active or passive.

With regard to the active fastener, this fastener may include an actuator (e.g., a linear or rotary actuator) operative under the control of the controller to tension the fastener to a desired tension. Sensors may be provided to sense the tension, generate corresponding sensor data and may provide this information to the controller for further processing such as to determine whether the fastener is closed, ready for operation, etc., and may render an indication of such on the UI for the convenience of the user (e.g., “fastener closed,” “close fastener,” “more tension needed,” “adjust fastener,” etc.). The controller may render at least one selection item on the UI that may be selected to toggle the fastener open or closed (e.g., by varying tension accordingly). For example, the fastener may include one or more sensors which may sense when it is fastened securely and may form a corresponding fastened sensor information and provide this information to the controller. The controller may then determine whether the fastener is fastened. The controller may activate the device when it is determined that the fastener is fastened. If the controller determines that the fastener is not securely fastened, the controller may deactivate the device to conserve power and may render indication of such on the UI for the convenience of the user. In some embodiments, the device may be activated at any time by depressing a power button which may transmit a corresponding signal to the controller which, in response, may activate the device.

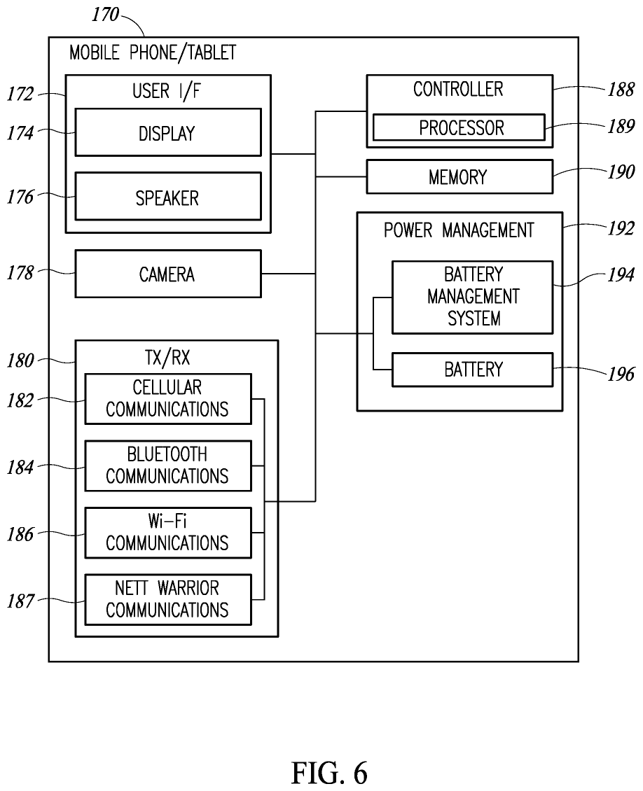

A block diagram illustrating a mobile station, phone, tablet, or other portable digital device (generally referred to as MS) of a multi-route countermeasure delivery system in more detail including example applications executing thereon is shown in FIG. 6. The portable digital device, generally referenced 170, may include one or more controllers 188, memory 190, image capture device such as a camera 178, power management block 192, UI 172, and transceiver (Tx/Rx) 180 one or more of which may communicate with each other using wired and/or wireless communication methods. The one or more controllers may each include at least one processor 189 such as a microprocessor or the like.

The power management block 192 may be similar to the power management block 146 (FIG. 5) of the wearable and may manage power throughout the MS. The power management block may include one or more power sources such as a capacitor and/or battery 196 configured to power the MS. A battery management system (BMS) 194 may be configured to manage charging and power draw of the battery. The BMS may monitor and/or charge the battery and/or individual cells therein. The BMS 194 may communicate with the BMS 148 (FIG. 5) of the device to obtain information related to the battery of the device such as health, charge, etc. and may provide this information to the one or more controllers 188 for rendering on the UI 172 for the convenience of the user. For example, the UI may render the information on a display 174 and/or a speaker 176. In some embodiments, BMS 194 and 148 may arrange for the transfer power therebetween so that a charge may be transferred between the battery 196 and 150 (FIG. 5) in either direction using wired and/or wireless power transfer method(s) when desired. Accordingly charging may be performed away without the need of mains power.

The UI may include at least one of the display 174 and the speaker 176 each of which may be configured to render information under the control of the one or more controllers. The display may include a touchscreen display configured to interact with the user via a touch interface. The speaker may be configured to render audio information under the control of the one or more controllers.

The camera 178 may be operative under the control of the one or more controllers and may be configured to capture an image, for corresponding image information, and transmit this image information to the one or more controllers for further processing, transmission via the Tx/Rx, rendering on the display, and/or storage in a memory of the system such as the memory for later use.

The Tx/Rx and may include one or more of a cellular communications portion 182, Bluetooth communications portion 184, Wi-Fi communications portion 186, and Nett Warrior communications portion 187 which may be configured to communicate with other portions of the system using cellular, Bluetooth, Wi-Fi, and Nett Warrior communication protocols, respectively.

Wireless communication methods may vary by device. For example, in some embodiments, the MS may communicate with the cloud server 62 (FIG. 4) using Wi-Fi, may communicate with other MSs of warfighters using cellular communications or the Nett Warrior network, may communicate with the device using Bluetooth. In accordance with some embodiments, the Tx/Rx may optionally include one or more of upconverters, downconverters, coder/decoders (CODECs), etc. Network protocols for transmission may be selected by users and/or by the system in accordance with available networks.

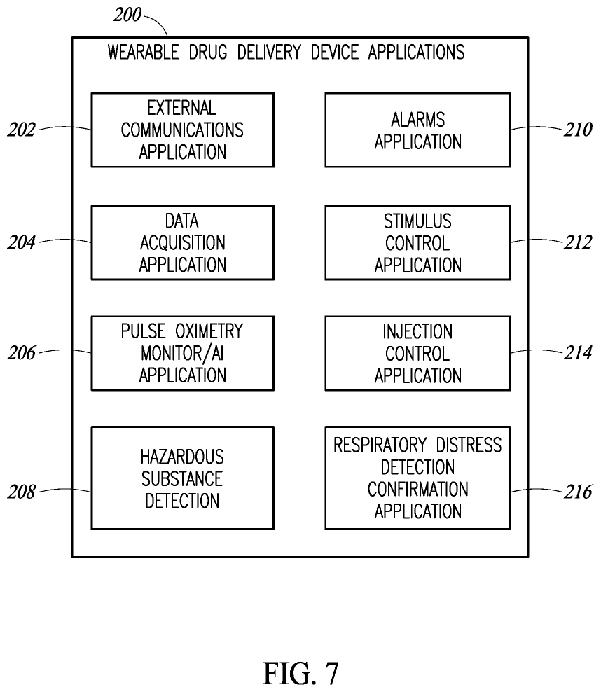

A block diagram illustrating an example wearable drug delivery device of a multi-route countermeasure delivery system in more detail including example applications executing thereon is shown in FIG. 7. The applications, generally referenced 200 may include an external communications application 202, data acquisition application 204, a pulse oximetry monitor/AI application 206, hazardous material detection 208, alarms application 210, stimulus (audile, haptic, shock, etc.) control application 212, injection control application 214, and respiratory distress detection/confirmation application 216, one or more of which may communicate and/or be combined with the other, and may be loaded, configured, and/or run by a controller of the system.

In some embodiments, at least one controller may load and/or run one or more of the applications upon activation of the device. In some embodiments, one or more of the applications may be run during an initial setup prior to activation and/or after activation, for example, to update system information and/or to store information in a memory of the system for later retrieval and/or use.

The external communications application 202 may be configured to establish and/or control communications between devices of one or more warfighters via Nett Warrior for establishing and communicating data and control.

The data acquisition application 204 may obtain sensor data from one or more sensors of the system such as such as pulse oximeter in contact with the skin of the warfighter. The data acquisition application may provide the acquired sensor data to the pulse oximetry monitor/AI application 206 for further processing. The data acquisition application may communicate with one or more available sensors to acquire sensor data therefrom. The sensors may be selected by availability. In some embodiments, the data acquisition application may form queries and transmit these queries to one or more sensors to obtain sensor data therefrom. In some embodiments, the data acquisition application may receive sensor data at periodic or non-periodic intervals. The sensor data may be pushed, pulled, etc.

The pulse oximetry monitor/AI application 206 may receive the sensor data, such as the PPGs, from the data acquisition application and may process the acquired data to form processed information, and may feed the processed information to an AI engine including an NN processor such as a CNN process, that it controls for further analysis in accordance with one or more AI models and/or algorithms obtained from a memory of the system. The AI engine may then output a prediction which represents a confidence value epsilon (ε) which may be a normalized, by a normalization engine, to a value that is between 0 and 1 and provide this value to the hazardous material detection application 208 for further analysis.

The hazardous material detection application 208 may determine a life threatening/respiratory distress situation in accordance with a value of E. The predication may include a discrete variable having one of J levels, where J is an integer and in the present embodiments may be set to three (although J may be set to other values) such that there may be J1, J2, and J3 levels of distress predictions which may correspond with a low likelihood, high likelihood, and an undetermined likelihood, respectively. Each of these J=3 levels may be set to have a corresponding threshold value or range of values (as shown in the present embodiments) associated therewith such that J1=0.00-0.33, J2=0.34-0.66, and J3=0.67-1.00. The hazardous material detection application may then compare the confidence value ε compared to the values of each of J1, J2 and J3 and determine whether the confidence value ε corresponds with the range of J1, J2 or J3. Accordingly, if it is determined that the confidence value ε corresponds with the range of J1, it may be determined that there is a high likelihood of exposure/distress and may set flag=1. If it is determined that the confidence value ε corresponds with the range of J2, it may be determined that there is a low likelihood of exposure/distress and may set flag=2. If it is determined that the confidence value ε corresponds with the range of J3, it may be determined that there is an unknown likelihood of exposure/distress and may set flag=3. Thus, each flag may represent one or more discrete levels as may be determined by the system. The results of the analysis which may include an exposure/distress prediction may include, for example, the flags and/or information associated therewith, may then be provided to a confirmation application for further analysis.

In some embodiments the discrete confidence ranges may be determined based upon a comparison of the confidence value ε with one or more threshold values. For example, assuming two discrete confidence ranges such as a high confidence range and a low confidence range separated by a confidence threshold value such as 0.50. The system may then determine whether the confidence value ε is less than or equal to the confidence threshold value. If it is determined that the confidence value ε is less than or equal to the confidence threshold value, the system may determine that there is a high confidence that the warfighter is in distress or unconscious and may set the flag=1 to indicate that there is a high likelihood of a problem. If it is determined that the confidence value ε is not less than or equal to the confidence threshold value (e.g., is greater than), the system may determine that there is a low confidence that the warfighter is in distress/unconscious and may set the flag=2 to indicate that there is a low likelihood. In some embodiments, the prediction may include the confidence value.

The OD detection confirmation application 216 may receive the predication from the hazardous material detection application 208, may render a challenge to interact with the warfighter in response thereto, and may determine whether they are experiencing distress/unconsciousness based upon a timely response to the challenge from the warfighter within a first threshold time period. If it is determined that a response to the challenge is not received within the first threshold time period, it may be determined that the warfighter is suffering from distress/unconsciousness and an indication of such may be generated and may be provided to the stimulus control application 212, injector control application 214, and/or alarm control application 210 for further analysis. If it is determined that a response to the challenge is received within the first threshold time period (i.e. is timely), the first threshold time period may be reset to its initial value, the challenge may be rendered once again, and the system may wait for a response to the challenge within the first threshold time period once again. This process may be repeated until a second time threshold is determined to have elapsed at which time associated information may be stored in a memory of the system for later use and/or analysis.

With regard to the challenge and the first and second time thresholds, in some embodiments, these may be selected, adjusted, and/or set/reset in accordance with the prediction. For example, the challenge may be selected from a plurality of challenges and/or one or more of the time thresholds (e.g., the first and second time thresholds) may be set in accordance with the predication. For example, if there is a high likelihood of distress/unconsciousness (as indicated by the prediction) the first threshold time period may be shorter than if there was a low likelihood of distress/unconsciousness. This may provide for a more immediate response when time is of the essence. Similarly, the second time threshold may be set to longer value. This may provide more time for monitoring the warfighter when the likelihood of a problem is higher.

Lastly, with regard to challenges, these may be assigned a likelihood of distress/unconsciousness. For example, there may be challenges that may be assigned to a low likelihood and others that may be assigned to a high likelihood. In some embodiments, the challenges and/or the time thresholds may be selected in accordance with the challenge while in other embodiments, the challenges and/or time thresholds may be preset and stored in a memory of the system for later selection and/or use.

The stimulus control application 212 may be configured to apply a physical stimulus to the warfighter using any suitable form of stimulation such as audible, haptic, chemical, mechanical or electrical (e.g., electric shock) upon receiving a signal from the respiratory distress detection confirmation application 216. Accordingly, the stimulus control application may receive at least one signal from the respiratory distress detection confirmation application. In response, the stimulus control application may control a high voltage electronic generator (e.g., a high voltage coil, etc.) to generate a low or high voltage signal which may be applied to the skin of the warfighter via a pair of electrodes coupled to the high voltage electronic generator and in contact with the skin thereof. The high voltage signal may have a duration corresponding with a shock threshold time and when applied to the skin may shock the warfighter to awaken and/or gain the attention thereof.

The stimulus may have any desired duration (e.g., two seconds, etc.), waveform shape (sine, step, ramp, etc.), amplitude, and/or frequency and may be repeated at periodic and/or nonperiodic intervals (e.g., a response threshold time such as every 20 seconds, etc.) until a response is received via UI of the MS of the warfighter and/or the device. If a response is not received within a response threshold time (e.g., 20 seconds, etc.) the shock may be repeated, etc.

The electrodes to deliver electric shock may include an adhesive coating configured to adhere to the skin of the warfighter and may include a conductive layer such as a gel to transfer the electrical signal to the skin.

The injection control application 214 may be configured to control an injector to provide at least one dose of a bioactive substance to the warfighter when respiratory distress/unconsciousness is confirmed. Accordingly, the injection control application may receive at least one signal from the hazardous material detection application and may analyze this signal to determine whether respiratory distress/unconsciousness is present. If not, the injection control application may continue to monitor signals received from the hazardous material detection application. If the signal is present, the injection control application may be configured to initiate an injection process to administer at least one dose of at least one bioactive substance to the warfighter. This injection process may vary based upon a type of injectors employed by the system which may include passive and active type injectors.

With regard to passive type injectors, an injection process may control at least one actuator configured to trigger the start of a corresponding injection process and may be powered by one or more of hydraulic, pneumatic, mechanical (e.g., springs), and/or electronic actuators. In some embodiments, an actuator may be controlled to control a depth of penetration of a hypodermic needle such that it may penetrate to a desired depth which may prevent the needle from striking a bone of the warfighter in the area of penetration.

With regard to active type injectors, the injection process may control one or more actuators to control position, direction, and/or speed (the latter being velocity) of the hypodermic needle and/or flow rate of a pump configured to pump the drug from a reservoir to the warfighter via the hypodermic needle to administer medication. One or more sensors may obtain sensor data which may be provided to a controller of the system for further analysis such as to determine one or more of direction and speed the hypodermic needle and/or flow rate of the pump and/or total volume of fluid displaced. The controller may then control the actuators in accordance with embodiments of the present system.

After the desired dose of the desire medication is administered (e.g., the total volume is equal to the desired dose volume) and/or upon receiving an abort signal during the injection process, the pump may be stopped and the hypodermic needle may be reversed to withdraw it from the skin of the warfighter and/or place it in a parked position. The speed of the hypodermic needle and/or dose pump as well as dose may vary in accordance with system settings. The injection process may be aborted at any time by the user.

Injection status may be monitored by the injection control application and may be rendered on a rendering device of the system such as a display of the device and/or MS of the warfighter. Injection status may include information related to an injection such as “Injection in process” “Injection completed at (3:00 pm), 4 mg Narcan administered,” “Injection Stopped,” etc.

When initiated, the injection process may control one or more actuators based upon a type of injector employed by the system such as a passive injector in which an injection process may be triggered and thereafter may run automatically, or an active injector in which hypodermic needle and/or medication pump displacement (speed and/or position) may be controlled in accordance feedback information (e.g., obtained from sensor data) and/or system settings. The one or more actuators may include one or more of linear or rotary actuators, linear or rotary motors, pneumatic actuators, spring actuators, etc., depending upon embodiments.

An abort option may be rendered on a rendering device such as the device and/or MS of the warfighter and, when selected (as may be determined by the system), the injection process may be stopped (e.g., pumps stopped, hypodermic needle withdrawn and parked) and an indication of such may be rendered on the rendering device for the convenience of the warfighter. Accordingly, the injector control application may determine whether the abort selection item has been selected by the user, and if it is determined that the abort selection item has been selected, the injection control application may be configured to stop the injection from occurring or to abort an injection in process (e.g., by stopping a pump and/or withdrawing the hypodermic needle from the user). Feedback sensor data may be provided to a controller of the system and information relating thereto may be rendered for the convenience of a user such as “Hypodermic Needle Withdrawn, Safe to Remove, 0.5 mg dose of administered” on a rendering device of the system such as on the display of the device or MS of the warfighter. Indication of user status such as “User alert,” “User unconscious,” etc. may also be rendered.

The alarm control application may be configured to render one or more alarms such as an user alarms upon receiving the signal from the respiratory distress detection confirmation application. If the input signal is detected, the alarm control application may generate, transmit, and/or render one or more alarms such as the user alarms to inform the user of the distress detection.

With regard to the alarms to inform the user, the alarm control application may generate and render one or more alarms which may be rendered on one or more rendering devices of the MS of the user and/or the device such as on a display, a speaker, a haptic device (e.g., a vibrator, etc.), an electrical stimulator, etc., to gain the user's attention and/or to awaken the user from sleep, to render instructions for the user to follow if possible, and/or to respond to a request for input such as “press here to respond.” Inputs from the user may then be relayed to a controller of the system for further analysis such as to determine whether the user is experiencing respiratory distress, is awake, etc. In some embodiments, prior to generating an electrical shock, the alarm control application may provide an electrical stimulation via the pair of electrodes employed by the stimulus control application.

With regard to the alarms to inform medical personnel/officer, they may be alerted via their MSs and/or contact information. Alerts may be updated in real time and be the same or customized for each recipient. In the present embodiments it will be assumed that each recipient may receive the same information related to the warfighter such as one or more of user ID, user status (“currently experiencing respiratory distress”), location, start time of the current event (distress detected 12:04 pm, 3 minutes ago, etc.), recipient contact information, sensor data including vitals, physiological information, current status (e.g., Naloxone/Nalmefene administered, breathing restored, etc.), etc.

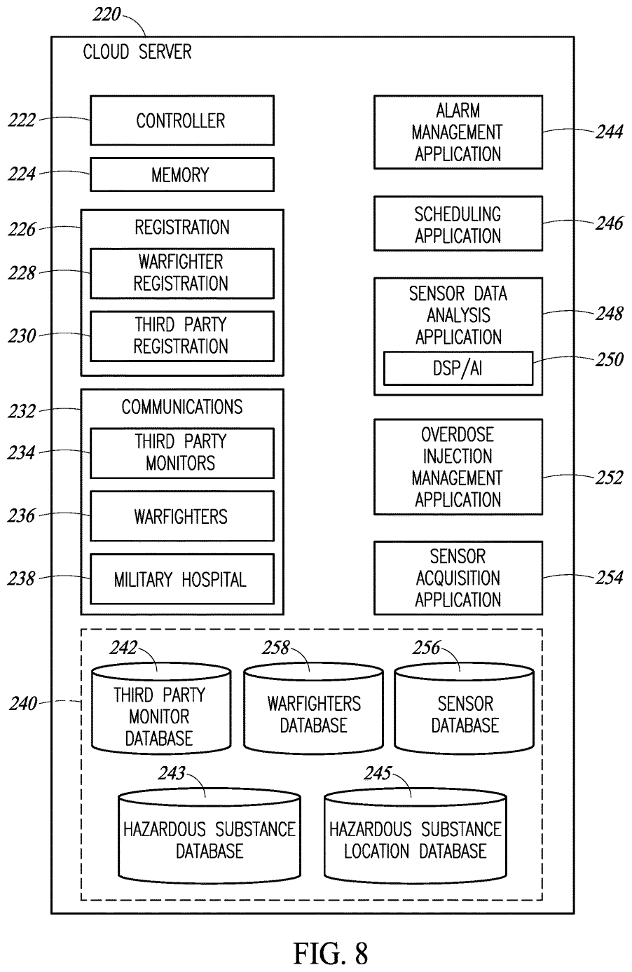

A block diagram illustrating an example cloud server of a multi-route countermeasure delivery system in more detail including example applications executing thereon and databases maintained therein is shown in FIG. 8. The cloud server (CS), generally referenced 220 comprises controller 222, memory 224, registration application 226 including warfighter registration 228 and third party registration 230, communications application 232 including third party monitors 234, warfighters 236, military hospital 238, alarm management application 244, scheduling application 246, sensor data analysis application 248 including DSP/AI module 250, injection management application 252, sensor acquisition application 254, databases 240 including third party monitor database 242, warfighter database 258, sensor database 256, hazardous substance database 243, and hazardous substance location database 245, one or more of which may communicate with each other via optical, wired, and/or wireless communication methods.

For the sake of clarity, the following description may be made to with reference to a single warfighter/user and the user's respective MS and device unless the context indicates otherwise. It is appreciated that embodiments of the present system may be operative with a plurality of user and respective MSs and devices.

The controller may control the overall operation of the CS and may load and/or run one or more applications such as one or more of the communications application, sensor acquisition application, alarm management application, scheduling application, sensor data analysis application, registration application, and injection management application. The controller may obtain operating instructions from a memory of the system, such as the memory 224 and may include any suitable memory which may be local to the CS and/or may distributed throughout the system. The controller may be configured to provide a UI with which one or more users of the system such as the warfighter, medical officer, commander, etc. may interact with the system.

The communications application 232 may control communications of the CS and may be configured to enable communication between, or with, one or more users of the system such as the warfighters via their respective MSs and/or via their respective contact information. The communication application may include one or more of a third party monitor communication 234 which may control communication to the monitors, warfighter communications 236 which may control communication to the warfighters, and military hospital communications 238 which may control communication to one or more military medical facilities. The communications application may control communication between the user and the respective third party monitors. Communication may be established via any suitable network or networks of the system such as a telephony network, a cellular network, the Internet, etc.

It is also envisioned that the communications application may enable communication between one or more portions of the CS such as one or more of the databases under the control of the controller to, for example, store information in and/or to retrieve information from the corresponding database.

The alarm management application may manage alarms of the system and may transmit alarms such as alerts and notifications to one or more portions of the system such as one or more of the device or MS of a user and any third parties under the control of the controller. For example, the alarm management application may provide notifications to the MSs such as the MS of the user and the MSs of third parties (medical officer, commander, etc.) in accordance with embodiments of the present system and when respiratory distress is detected, the alarm management application may transmit an alarm to one or more of the third parties and provide information related to the user such as user ID, location, etc.

The sensor acquisition application may acquire sensor information from one or more sensors of the system, such as sensors of a user's device or corresponding MS and may provide the acquired sensor information to the controller and/or the sensor data analysis application 248 for further analysis processing and/or storage in a memory of the system such as memory 224. In some embodiments, the acquired sensor information may be processed or preprocessed by the controller or may be provided directly to the sensor data analysis application for further analysis and processing.

For example, it is envisioned that the controller may process SpO₂ sensor information to form PPG sensor information which may then be provided to the sensor data analysis application for further processing. The sensor acquisition application 254 may obtain information related to each user of the system and/or groups and/or subgroups of users and may obtain sensor information related to each (or selected groups or subgroups) user from their respective MS and/or device.

The sensor data analysis application may include a DSP/AI application 250 and may obtain sensor information from one or more sensors of the system such as PPG information, and may process the sensor information using the DSP/AI application to obtain one or more predications in accordance with embodiments of the present system. These predications may then be further processed with sensor information to determine whether the user is experiencing respiratory distress and/or the likelihood thereof. Results of the determination may then be provided to the controller and/or one or more MSs of the system such as MSs of the user, third parties, etc., in accordance with embodiments of the present system.

For example, analysis results may be returned to the MSs of the user and/or third parties associated therewith. The sensor data analysis application may further generate and/or update one or more AI models, machine learning models, and/or algorithms and store them in a memory of the system such as the memory for later use. The sensor data analysis application may preprocess sensor information to be processed by the DSP/AI application and may acquire AI models and/or algorithms to be applied to DSP/AI analysis by the DSP/AI application when processing sensor information generated by the system. The preprocessing may include, for example, processing pulse oximetry sensor information such as SpO₂ to form corresponding PPGs that may be processed by the DSP/AI application. The sensor data analysis application may obtain algorithms and/or models from one or more memories of the system and may obtain sensor information in real time and/or from one or more memories of the system.

The injection management application 252 manages the injection process of the user when it is determined that the user is experiencing respiratory distress. The injection management may further control a stimulation process that may provide a stimulus to be applied to the user such as audible, haptic, electric shock, chemical stimulus, etc. when it is determined that the user is in respiratory distress. The injection management application may generate a signal waveform to be applied to the user and may control the user's device in an attempt to awaken the user. The injection management application, may interact with a user to inform of a pending injection and/or shock, and may provide a selection item for by a user of the system to abort the injection. The injection management application may provide a status of an injection administered to the user to one or more users of the system such as third parties via their respective MSs and may manage an information exchange between one or more of the user and third parties when it is determined that the user is experiencing respiratory distress.

The third party monitor database may include information related to one or more of the third parties such as identification (ID), contact address, schedule information (e.g., schedule of availability), current availability (e.g., currently online, offline, etc.), history, relationship to the user, authentication codes (e.g., access codes), and authentication settings (access levels, access databases, etc.).

The registration application 226 may provide registration services for warfighters and/or third parties which may thereafter be used for authentication. Registration may be performed on a one-time basis with no identifiable information related to one or more of the user and/or third party or may be stored for later use and/or authentication. Authentication may be performed using an ID unique to a user which ID may include email addresses, telephone numbers, social networking addresses, unique IDs, passwords, etc. Once authenticated, the system may be configured to provide access to the system in accordance with access rights for each user.

It is envisioned that the registration application may provide a user interface (UI), such as a GUI or the like, with which a user may interact with the system and/to perform a registration for the same of another user or third party. The registration application may include one or more of a warfighter registration application 228 and a third party registration application 230.

The user registration application may provide one or more applications configured to register a warfighter to the system. The registration may be performed at each use or may be performed once and thereafter may be automatic each time the MS of the user uses an application of the system or certain actions are sensed (e.g., wearable is worn, injector cartridge coupled or removed, etc.). In some embodiments, the user may use an MS which is not registered to the user and may perform a registration using authentication information specific to the corresponding user such as one or more of an account name, ID, email account, biometric-ID information, social networking account, and/or password, etc. pre-registered to the system for the user. These settings may be stored in a memory of the system. Once registered, the user may use one or more functions of the system in accordance with system settings.

The third party registration application may provide one or more applications which may be configured to register a third party to the system. The registration may be performed at each use or may be performed once and thereafter may be automatic each time the MS of the third party uses an application of the system. In some embodiments, the third party may use an MS which is not registered thereto and may perform a registration using authentication information specific to the corresponding third party such as one or more of an account name, ID, email account, social networking account, and/or password, etc., which may be pre-registered to the system for the third party.

The warfighter database may include information related to one or more of the users such as identification (ID), contact address, working address, corresponding third parties such as medical officer, commanders, etc., current operational status (e.g., currently online, offline, etc.), history (e.g., drug use, past events, etc.), authentication codes (e.g., access codes), and setting information.

The sensor database may include information related to one or more sensors of the system such as settings, accuracy, type (e.g., reflective pulse oximeter sensor, etc.), identification (ID), calibration settings, parameters, connection preferences (e.g., Bluetooth, etc.), pairing information, etc. The sensor database may further include sensor information generated by one or more sensors of the system.

The CS also comprises a hazardous substance database listing known potential hazardous substances the warfighter can potentially encounter as well as their known or likely locations.

It is appreciated that alternate layout and position of features of the device from that shown in the FIGS. 1-8 are also contemplated and within the scope of the present invention. For example, the device can be designed to load drug cartridges from the front side of the device rather than the rear of the device. In addition, a display may be included as part of the user interface to aid in conveying information to the user.

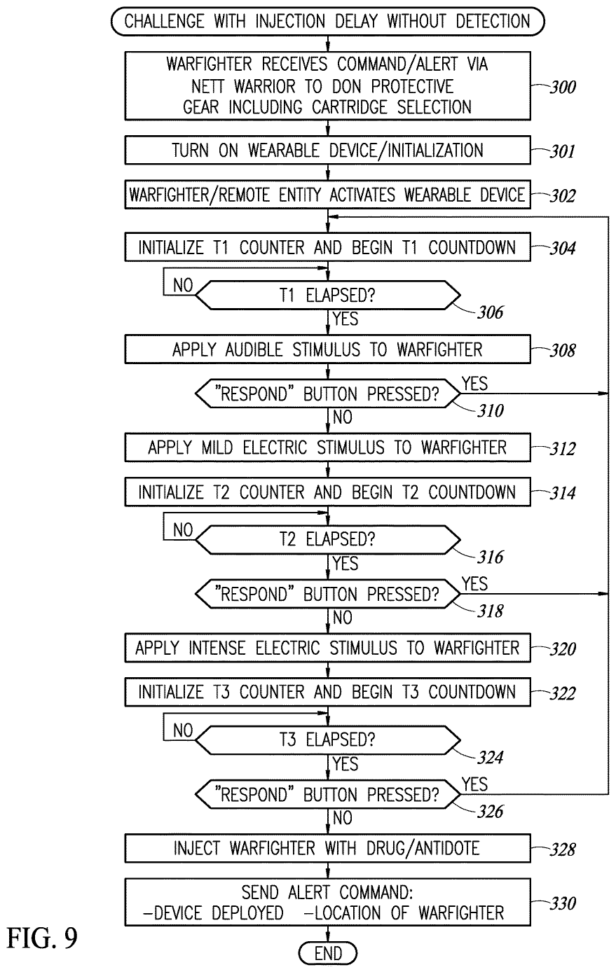

A flow diagram illustrating example distress detection and antidote delivery utilizing a challenge with injection delay without hazardous substance detection is shown in FIG. 9. The method begins when the warfighter (i.e. user) receives a command or alert locally, manually, or via the Nett Warrior system to don protective gear including cartridge selection (step 300). The command is recognized by the device controller and interpreted as a request to turn the device on for potential drug delivery. In response to the command/alert, the device is turned on and enters initialization (step 301) wherein the controller (1) verifies the device is in an operational state (power sufficient, no critical errors); (2) checks sensor status to confirm electrodes are properly attached; (3) initializes all peripheral circuits required for drug delivery; and (4) begins recording a timestamp for the delivery event.

The warfighter or third party/remote entity then activates the wearable device (step 302). The device then initializes the T1 counter to zero or a nominal starting value and begins counting (step 304). This counter will track elapsed time for delivery of a first stimulus to the warfighter in response to a failed first challenge. The T1 counter begins counting down at a fixed rate while optionally providing feedback via the user interface (display, speaker, etc.) indicating the countdown is in progress. Once the T1 count elapsed (step 306), a first stimulus is applied to the warfighter (step 308). In one example embodiment, the first stimulus comprises an audible alert. The user must then respond to the challenge in any suitable manner. In one example embodiment, the user presses the “RESPOND” button (step 310). If the user responds, the method returns to step 304 whereby the T1 counter is re-initialized and count down begins again. If the user does not respond (i.e. within a predetermined timeout interval of e.g., 30-60 seconds), a second stimulus is applied to the user, which may comprise a mild electric shock (step 312). The T1 countdown is part of the basic loop when the user responds to the audible stimulus. As long as the user keeps responding to the audible stimulus, the device remains in this loop.

Once the user fails to respond to the audible stimulus (i.e. the user is in respiratory distress or loses consciousness), the device applies a mild electric stimulus to the user (step 312) and initializes a second T2 counter and begins counting down (step 314). In one embodiment, the stimulus circuit in the device is operative to generate a signal having a voltage between 50-150V (non-therapeutic, sensory stimulus only), pulse duration of 10-50 microseconds, pulse frequency of 1-5 Hz, stimulus duration of 0.5-1.0 seconds, and energy delivery less than 0.1 mJ (below activation threshold for cell electroporation or electrokinetic transport). In this case, the user experiences a mild tingling or slight electrical sensation at the electrode site.

Once the T2 counter has elapsed (step 316), the device waits for the user to respond by pressing the “RESPOND” button (step 318). If the user responds, the method returns to step 304. Note that the RESPOND button may comprise a soft button on a touch screen, a dedicated hard mechanical button on the face of the device, or any other suitable user input facility.

If the user fails to respond to the second challenge, a more intense electric shock is generated and applied to the user (step 320). With this third stimulus, the user experiences a stronger electrical sensation (tingling, warmth) at the electrode site than the second stimulus. The stimulus characteristics in this example include a voltage level of 200-600V, a pulse peak current of 100-500 mA (limited by safety current-limiting resistors), energy per pulse of 0.5-2.0 mJ, and total energy delivered about 0.5-6.0 mJ.

A third counter T3 is initialized and begins counting down (step 322). Once the T3 counter elapses (step 324), the device waits for the user to respond to the third challenge and press the “RESPOND” button (step 326). If the user responds to the challenge, the method returns to step 304. Otherwise, the injector mechanism is activated and the warfighter is injected with one or more bioactive substances in response to the failed challenge (step 328). The bioactive substance may comprise any drug, antidote, non-drug, etc. An alert command indicating that the device deployed and injected the warfighter as well as the location of the warfighter's location is sent to appropriate entities (step 330).

In one example embodiment, the alert command includes (1) confirmation that the bioactive substance was mechanically dispensed (injector position verification); (2) confirmation that electrical stimulus was applied; (3) cross-validation of sensor readings during delivery; and (4) detailed event log for clinical records associated with the warfighter.

In one example embodiment, the injector comprises (1) a stepper motor or solenoid actuator to drive the plunger mechanism, (2) a syringe or cartridge barrel containing the bioactive substance, (3) a plunger operative to mechanically displace the bioactive substance from the reservoir, and (4) an optional pressure sensor to confirm drug release by monitoring pressure rise during actuation.

In one example embodiment, the delivery actuation sequence comprises (1) the controller signals the injector motor to move to a calculated position corresponding to the desired dose; (2) a plunger displacement drives pharmaceutical through the delivery catheter; (3) a pressure sensor (if present) confirms pharmaceutical movement; (4) delivery continues for the duration of the therapeutic stimulus application (synchronized release); and (5) the motor receives stop command when delivery is complete.

The dose volume is determined by plunger displacement distance (mm)×barrel cross-sectional area (mm²)=volume (mL). Controller firmware pre-calculates the required plunger displacement based on (1) user-specified dose (e.g., 0.1 mL, 0.5 mL, 1.0 mL); (2) reservoir fill level (measured by optical or electrical means); and (3) pharmaceutical viscosity (affects actuation force and timing).

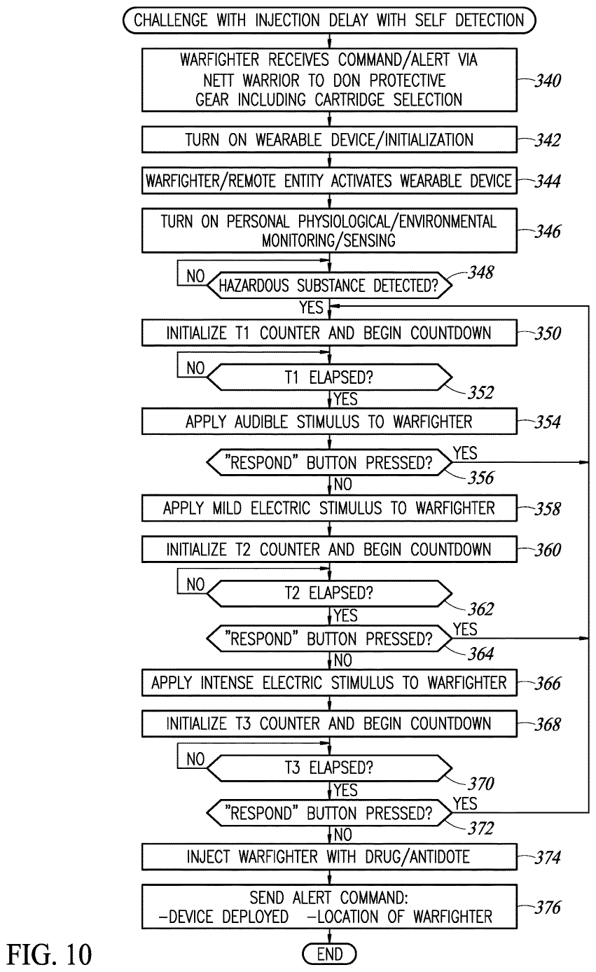

A flow diagram illustrating example distress detection and antidote delivery utilizing a challenge with injection delay with personal hazardous substance detection is shown in FIG. 10. The method begins when the warfighter (i.e. user) receives a command or alert locally, manually, or via the Nett Warrior system to don protective gear including cartridge selection (step 340). The command is recognized by the device controller and interpreted as a request to turn the device on for potential drug delivery. In response to the command/alert, the device is turned on and enters initialization (step 342) wherein the controller (1) verifies the device is in an operational state (power sufficient, no critical errors); (2) checks sensor status to confirm electrodes are properly attached; (3) initializes all peripheral circuits required for drug delivery; and (4) begins recording a timestamp for the delivery event.

The warfighter or third party/remote entity then activates the wearable device (step 344). The device then turns on (enables) physiological/environmental monitoring/sensing (step 346). The sensors incorporated in the device are activated and the environment and physiological status of the warfighter are monitored. Monitoring continues until a hazardous substance is detected (i.e. local alert) (step 348).

Once a hazardous substance is detected, the method continues similarly to the method of FIG. 9. In particular, the device initializes the T1 counter to zero or a nominal starting value and begins counting (step 350). This counter will track elapsed time for delivery of a first stimulus to the warfighter in response to a failed first challenge. The T1 counter begins counting down at a fixed rate while optionally providing feedback via the user interface (display, speaker, etc.) indicating the countdown is in progress. Once the T1 count elapsed (step 352), a first stimulus is applied to the warfighter (step 354). In one example embodiment, the first stimulus comprises an audible alert. The user must then respond to the challenge in any suitable manner. In one example embodiment, the user presses the “RESPOND” button (step 356). If the user responds, the method returns to step 350 whereby the T1 counter is re-initialized and count down begins again. If the user does not respond (i.e. within a predetermined timeout interval of e.g., 30-60 seconds), a second stimulus is applied to the user, which may comprise a mild electric shock (step 358). The T1 countdown is part of the basic loop when the user responds to the audible stimulus. As long as the user keeps responding to the audible stimulus, the device remains in this loop.

Once the user fails to respond to the audible stimulus (i.e. the user is in respiratory distress or loses consciousness), the device applies a mild electric stimulus to the user (step 358) and initializes a second T2 counter and begins counting down (step 360). In one embodiment, the stimulus circuit in the device is operative to generate a signal having a voltage between 50-150V (non-therapeutic, sensory stimulus only), pulse duration of 10-50 microseconds, pulse frequency of 1-5 Hz, stimulus duration of 0.5-1.0 seconds, and energy delivery less than 0.1 mJ (below activation threshold for cell electroporation or electrokinetic transport). In this case, the user experiences a mild tingling or slight electrical sensation at the electrode site.

Once the T2 counter has elapsed (step 362), the device waits for the user to respond by pressing the “RESPOND” button (step 364). If the user responds, the method returns to step 350. Note that the RESPOND button may comprise a soft button on a touch screen, a dedicated hard mechanical button on the face of the device, or any other suitable user input facility.

If the user fails to respond to the second challenge, a more intense electric shock is generated and applied to the user (step 366). With this third stimulus, the user experiences a stronger electrical sensation (tingling, warmth) at the electrode site than the second stimulus. The stimulus characteristics in this example include a voltage level of 200-600V, a pulse duration of 10-100 microseconds, pulse frequency of 1-10 Hz, train duration of 1-3 seconds, peak current of 100-500 mA (limited by safety current-limiting resistors), energy per pulse of 0.5-2.0 mJ, and total energy delivered about 0.5-6.0 mJ.

A third counter T3 is initialized and begins counting down (step 368). Once the T3 counter elapses (step 370), the device waits for the user to respond to the third challenge and press the “RESPOND” button (step 372). If the user responds to the challenge, the method returns to step 350. Otherwise, the injector mechanism is activated and the warfighter is injected with one or more bioactive substances in response to the failed challenge (step 374). The bioactive substance may comprise any drug, antidote, non-drug, etc. An alert command indicating that the device deployed and injected the warfighter as well as the location of the warfighter's location is sent to appropriate entities (step 376).

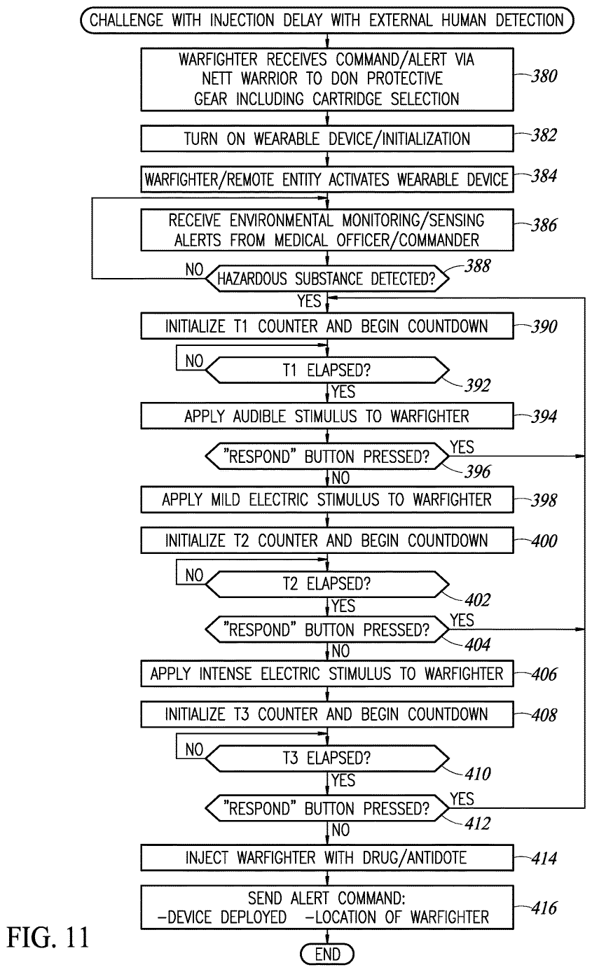

A flow diagram illustrating example distress detection and antidote delivery utilizing a challenge with injection delay with external human hazardous substance alert is shown in FIG. 11. The method begins when the warfighter (i.e. user) receives a command or alert locally, manually, or via the Nett Warrior system to don protective gear including cartridge selection (step 380). The command is recognized by the device controller and interpreted as a request to turn the device on for potential drug delivery. In response to the command/alert, the device is turned on and enters initialization (step 382) wherein the controller (1) verifies the device is in an operational state (power sufficient, no critical errors); (2) checks sensor status to confirm electrodes are properly attached; (3) initializes all peripheral circuits required for drug delivery; and (4) begins recording a timestamp for the delivery event.

The warfighter or third party/remote entity then activates the wearable device (step 384). The device receives environmental monitoring/sensing alerts from a medical officer or commander (remote alert) (step 386). Monitoring of the alerts continues until an alert is received indicating a hazardous substance is detected (step 388).

Once a hazardous substance is detected, the method continues similarly to the method of FIG. 9. In particular, the device initializes the T1 counter to zero or a nominal starting value and begins counting (step 390). This counter will track elapsed time for delivery of a first stimulus to the warfighter in response to a failed first challenge. The T1 counter begins counting down at a fixed rate while optionally providing feedback via the user interface (display, speaker, etc.) indicating the countdown is in progress. Once the T1 count elapsed (step 392), a first stimulus is applied to the warfighter (step 394). In one example embodiment, the first stimulus comprises an audible alert. The user must then respond to the challenge in any suitable manner. In one example embodiment, the user presses the “RESPOND” button (step 396). If the user responds, the method returns to step 390 whereby the T1 counter is re-initialized and count down begins again. If the user does not respond (i.e. within a predetermined timeout interval of e.g., 30-60 seconds), a second stimulus is applied to the user, which may comprise a mild electric shock (step 398). The T1 countdown is part of the basic loop when the user responds to the audible stimulus. As long as the user keeps responding to the audible stimulus, the device remains in this loop.

Once the user fails to respond to the audible stimulus (i.e. the user is in respiratory distress or loses consciousness), the device applies a mild electric stimulus to the user (step 398) and initializes a second T2 counter and begins counting down (step 400). In one embodiment, the stimulus circuit in the device is operative to generate a signal having a voltage between 50-150V (non-therapeutic, sensory stimulus only), pulse duration of 10-50 microseconds, pulse frequency of 1-5 Hz, stimulus duration of 0.5-1.0 seconds, and energy delivery less than 0.1 mJ (below activation threshold for cell electroporation or electrokinetic transport). In this case, the user experiences a mild tingling or slight electrical sensation at the electrode site.

Once the T2 counter has elapsed (step 402), the device waits for the user to respond by pressing the “RESPOND” button (step 404). If the user responds, the method returns to step 390. Note that the RESPOND button may comprise a soft button on a touch screen, a dedicated hard mechanical button on the face of the device, or any other suitable user input facility.

If the user fails to respond to the second challenge, a more intense electric shock is generated and applied to the user (step 406). With this third stimulus, the user experiences a stronger electrical sensation (tingling, warmth) at the electrode site than the second stimulus. The stimulus characteristics in this example include a voltage level of 200-600V, a pulse peak current of 100-500 mA (limited by safety current-limiting resistors), energy per pulse of 0.5-2.0 mJ, and total energy delivered about 0.5-6.0 mJ.

A third counter T3 is initialized and begins counting down (step 488). Once the T3 counter elapses (step 410), the device waits for the user to respond to the third challenge and press the “RESPOND” button (step 412). If the user responds to the challenge, the method returns to step 390. Otherwise, the injector mechanism is activated and the warfighter is injected with one or more bioactive substances in response to the failed challenge (step 414). The bioactive substance may comprise any drug, antidote, non-drug, etc. An alert command indicating that the device deployed and injected the warfighter as well as the location of the warfighter's location is sent to appropriate entities (step 416).

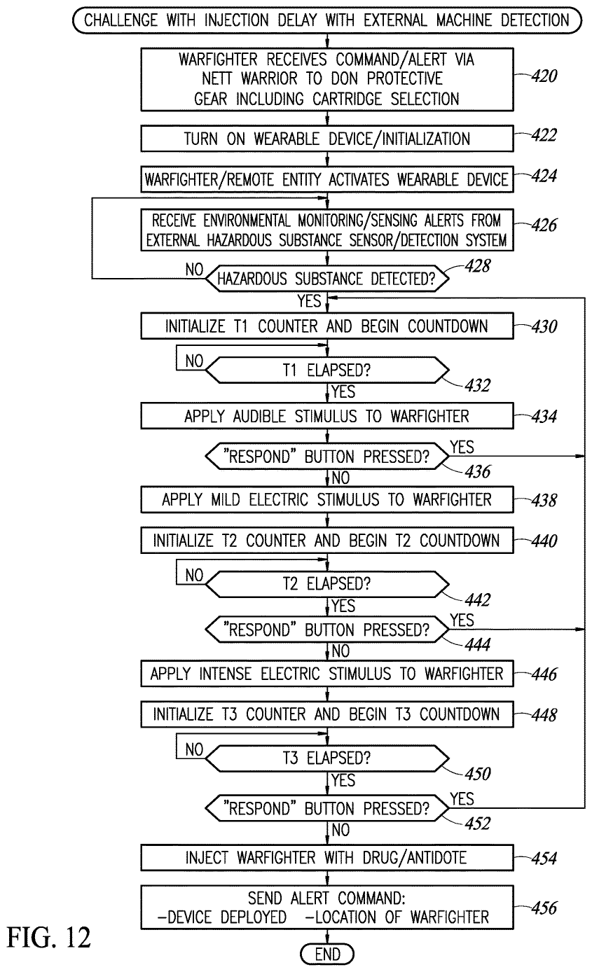

A flow diagram illustrating example distress detection and antidote delivery utilizing a challenge with injection delay with external machine hazardous substance monitoring and alert is shown in FIG. 12. The method begins when the warfighter (i.e. user) receives a command or alert locally, manually, or via the Nett Warrior system to don protective gear including cartridge selection (step 420). The command is recognized by the device controller and interpreted as a request to turn the device on for potential drug delivery. In response to the command/alert, the device is turned on and enters initialization (step 422) wherein the controller (1) verifies the device is in an operational state (power sufficient, no critical errors); (2) checks sensor status to confirm electrodes are properly attached; (3) initializes all peripheral circuits required for drug delivery; and (4) begins recording a timestamp for the delivery event.

The warfighter or third party/remote entity then activates the wearable device (step 424). The device receives environmental monitoring/sensing alerts from an external/remote hazardous substance sensor/detection system (remote alert) (step 426). Monitoring of the alerts continues until an alert is received indicating a hazardous substance was detected (step 428).

Once a hazardous substance is detected, the method continues similarly to the method of FIG. 9. In particular, the device initializes the T1 counter to zero or a nominal starting value and begins counting (step 430). This counter will track elapsed time for delivery of a first stimulus to the warfighter in response to a failed first challenge. The T1 counter begins counting down at a fixed rate while optionally providing feedback via the user interface (display, speaker, etc.) indicating the countdown is in progress. Once the T1 count elapsed (step 432), a first stimulus is applied to the warfighter (step 434). In one example embodiment, the first stimulus comprises an audible alert. The user must then respond to the challenge in any suitable manner. In one example embodiment, the user presses the “RESPOND” button (step 436). If the user responds, the method returns to step 430 whereby the T1 counter is re-initialized and count down begins again. If the user does not respond (i.e. within a predetermined timeout interval of e.g., 30-60 seconds), a second stimulus is applied to the user, which may comprise a mild electric shock (step 438). The T1 countdown is part of the basic loop when the user responds to the audible stimulus. As long as the user keeps responding to the audible stimulus, the device remains in this loop.

Once the user fails to respond to the audible stimulus (i.e. the user is in respiratory distress or loses consciousness), the device applies a mild electric stimulus to the user (step 438) and initializes a second T2 counter and begins counting down (step 440). In one embodiment, the stimulus circuit in the device is operative to generate a signal having a voltage between 50-150V (non-therapeutic, sensory stimulus only), pulse duration of 10-50 microseconds, pulse frequency of 1-5 Hz, stimulus duration of 0.5-1.0 seconds, and energy delivery less than 0.1 mJ (below activation threshold for cell electroporation or electrokinetic transport). In this case, the user experiences a mild tingling or slight electrical sensation at the electrode site.

Once the T2 counter has elapsed (step 442), the device waits for the user to respond by pressing the “RESPOND” button (step 444). If the user responds, the method returns to step 430. Note that the RESPOND button may comprise a soft button on a touch screen, a dedicated hard mechanical button on the face of the device, or any other suitable user input facility.

If the user fails to respond to the second challenge, a more intense electric shock is generated and applied to the user (step 446). With this third stimulus, the user experiences a stronger electrical sensation (tingling, warmth) at the electrode site than the second stimulus. The stimulus characteristics in this example include a voltage level of 200-600V, a pulse duration of 10-100 microseconds, pulse frequency of 1-10 Hz, train duration of 1-3 seconds, peak current of 100-500 mA (limited by safety current-limiting resistors), energy per pulse of 0.5-2.0 mJ, and total energy delivered about 0.5-6.0 mJ.

A third counter T3 is initialized and begins counting down (step 448). Once the T3 counter elapses (step 450), the device waits for the user to respond to the third challenge and press the “RESPOND” button (step 452). If the user responds to the challenge, the method returns to step 430. Otherwise, the injector mechanism is activated and the warfighter is injected with one or more bioactive substances in response to the failed challenge (step 454). The bioactive substance may comprise any drug, antidote, non-drug, etc. An alert command indicating that the device deployed and injected the warfighter as well as the location of the warfighter's location is sent to appropriate entities (step 456).

Those skilled in the art will recognize that the boundaries between logic and circuit blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediary components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

In the claims, any reference signs placed between parentheses shall not be construed as limiting the claim. The use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first,” “second,” etc. are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. As numerous modifications and changes will readily occur to those skilled in the art, it is intended that the invention not be limited to the limited number of embodiments described herein. Accordingly, it will be appreciated that all suitable variations, modifications and equivalents may be resorted to, falling within the spirit and scope of the present invention. The embodiments were chosen and described in order to best explain the principles of the invention and the practical application, and to enable others of ordinary skill in the art to understand the invention for various embodiments with various modifications as are suited to the particular use contemplated.

What is claimed is:

1. A method of detecting and treating hazardous substance exposure of a warfighter for use in a wearable countermeasure system, the method comprising:
rendering a plurality of interactive challenges to the warfighter;
continuing to present challenges to the warfighter as long as the warfighter successfully passes said challenges;
upon failing a challenge, applying a stimulus to the warfighter and continuing to present said challenges; and
upon failing a predetermined number of challenges, initiating injection of one or more bioactive substances to the warfighter.

2. The method according to claim 1, wherein said interactive challenge comprises having the warfighter complete a task on said wearable countermeasure system, said task comprising solving a puzzle, playing a game, pressing a physical button, pressing a switch, or pressing a virtual button.

3. The method according to claim 1, wherein said one or more bioactive substances comprises naloxone, Narcan, naltrexone, nalmefene, buprenorphine, Atropine, Pralidoxime Chloride, opioid antagonist, a pharmacological agent, chemical warfare agent antidote, opioid antidote, nutritional supplement, vitamin, mineral, electrolyte, adaptogen, hormone precursor, performance-enhancing compound, diagnostic agent, or any combination thereof.

4. The method according to claim 1, further comprising providing a periodic external stimulus to the warfighter after injection of said one or more bioactive substances, said stimulus cancellable thereby.

5. A method of detecting and treating hazardous substance exposure of a warfighter for use in a wearable countermeasure system, the method comprising:
receiving an alert indicating exposure or potential exposure to hazardous material;
in response to the alert, rendering a plurality of interactive challenges to the warfighter;
continuing to present challenges to the warfighter as long as the warfighter successfully passes said challenges;
upon failing a challenge, applying a stimulus to the warfighter and continuing to present said challenges; and
upon failing a predetermined number of challenges, initiating injection of one or more bioactive substances to the warfighter.

6. The method according to claim 5, further comprising monitoring the warfighter for hazardous material exposure utilizing one or more sensors and generating said alert in the event hazardous material exposure is detected.

7. The method according to claim 6, wherein said one or more sensors comprises at least one of a chemical sensor, radiological sensor, pulse oximeter sensor, end tidal CO2 sensor, chest wall muscle monitor, capnography, contactless respiratory rate monitor, body temperature monitor, infrared sensor, heart rate monitor, blood pressure monitor, motion sensor, radar based sensor, optical based sensor, acoustic sensor, thermal based sensor, a respiration monitor belt, and a respiratory sensor.

8. The method according to claim 6, wherein said monitoring comprises obtaining pulse oximetry information from a pulse oximetry sensor in contact with the warfighter.

9. The method according to claim 5, further comprising receiving said alert indicating exposure or potential exposure to hazardous material from an external human source including a commander, medical officer, combat medic, medical service corps personnel, and/or medical warrant officer.

10. The method according to claim 5, further comprising receiving said alert indicating exposure or potential exposure to hazardous material from an external hazardous material sensor/detector in communication with the wearable countermeasure system.

11. The method according to claim 5, wherein said challenge comprises having the warfighter complete a task on said wearable countermeasure system, said task comprising solving a puzzle, playing a game, pressing a physical button, pressing a switch, or pressing a virtual button.

12. The method according to claim 5, wherein said one or more bioactive substances comprises naloxone, Narcan, naltrexone, nalmefene, buprenorphine, Atropine, Pralidoxime Chloride, opioid antagonist, a pharmacological agent, chemical warfare agent antidote, opioid antidote, nutritional supplement, vitamin, mineral, electrolyte, adaptogen, hormone precursor, performance-enhancing compound, diagnostic agent, or any combination thereof.

13. The method according to claim 5, further comprising providing a periodic external stimulus to the warfighter after injection of said one or more bioactive substances, said stimulus cancellable thereby.

14. A wearable countermeasure system for detecting and treating hazardous substance exposure of a warfighter, comprising:
a housing;
a user interface for accepting one or more user commands and displaying information;
an injector assembly operative to inject the warfighter with one or more bioactive substances in response to an inject command;
a stimulus unit operative to generate a stimulus to be applied to the warfighter;
a controller comprising instructions stored in a memory and operative to:
receive an alert indicating exposure or potential exposure to a hazardous substance;
in response to the alert, render a plurality of interactive challenges to the warfighter;
continue to present challenges to the warfighter as long as the warfighter successfully passes said challenges;
upon failing a challenge, apply a stimulus to the warfighter and continue to present said challenges; and
upon failing a predetermined number of challenges, generate the inject command to inject one or more bioactive substances to the warfighter.

15. The apparatus according to claim 14, wherein said alert is generated by a sensor/detector in the event hazardous material exposure of the warfighter is detected.

16. The apparatus according to claim 14, wherein said alert is generated by from an external human source including a commander, medical officer, combat medic, medical service corps personnel, and/or medical warrant officer.

17. The apparatus according to claim 14, wherein said alert is generated by an external hazardous material sensor/detector in communication with the wearable countermeasure system.

18. The apparatus according to claim 14, wherein said challenge comprises having the warfighter complete a task on said wearable countermeasure system, said task comprising solving a puzzle, playing a game, pressing a physical button, pressing a switch, or pressing a virtual button.

19. The apparatus according to claim 14, wherein said one or more bioactive substances comprises naloxone, Narcan, naltrexone, nalmefene, buprenorphine, Atropine, Pralidoxime Chloride, opioid antagonist, a pharmacological agent, chemical warfare agent antidote, opioid antidote, nutritional supplement, vitamin, mineral, electrolyte, adaptogen, hormone precursor, performance-enhancing compound, diagnostic agent, or any combination thereof.

20. The apparatus according to claim 14, wherein said stimulus unit is operative to generate the stimulus to the warfighter after injection of said one or more bioactive substances, said stimulus cancellable thereby.
